(19) United States
(12) Patent Application Publication
(10) Pub. No.: US 2026/0207519 A1
(43) Pub. Date: Jul. 23, 2026

(54) ISOLATED TARGETED DELIVERY SYSTEM FOR THE TREATMENT OF OVARIAN CANCER

(71) Applicant: CELLIS SP. Z O.O. [LTD.], Warsaw (PL)

(72) Inventors: Magdalena KROL, Warsaw (PL); Tomasz RYGIEL, Warsaw (PL); Jan BRANCEWICZ, Warsaw (PL); Konrad GABRUSIEWICZ, Warsaw (PL); Waldemar LIPINSKI, Warsaw (PL); Ilona MARSZALEK, Warsaw (PL); Bartlomiej TACIAK, Warsaw (PL)

(21) Appl. No.: 19/140,875

(22) PCT Filed: Dec. 20, 2023
(86) PCT No.: PCT/EP2023/087070

§ 371 Date: Jun. 18, 2025

(30) Foreign Application Priority Data
Dec. 20, 2022 (EP) 22461645.8

(51) Int. Cl. A61K 9/50; A61K 31/4745; A61K 38/08; A61K 47/64; A61P 35/00; C12N 5/0786

(52) U.S. Cl. CPC A61K 9/5068; A61K 31/4745; A61K 38/08; A61K 47/644; A61K 47/6445; A61P 35/00; C12N 5/0645; C12N 2506/115

(57) ABSTRACT

The present invention relates to an isolated targeted delivery system comprising a CD45+ leukocyte cell comprising within said cell a complex of one or more iron binding proteins and a pharmaceutically active substance, label or pharmaceutically active substance and label, for use in a method of treatment or diagnosis of ovarian cancer.

The present invention relates to an isolated targeted delivery system comprising a CD45⁺ leukocyte cell comprising a complex of an iron binding protein and a pharmaceutically active substance and/or label, for use in a method of treatment or diagnosis of ovarian cancer.

### BACKGROUND OF THE INVENTION

About 300,000 women are diagnosed with ovarian cancer each year and the number of deaths is estimated at 180,000 worldwide each year. Ovarian cancer metastasizes early in its development, often before it is diagnosed. More than 60% of women with ovarian cancer are diagnosed with stage III or IV once it has spread beyond the ovaries. Ovarian cancer treatment involves surgery with frontline platinum-based chemotherapy. After relapse, patients receive second-line platinum-based chemotherapy and based on the outcome: PARP inhibitor or bevacizumab (Avastin) maintenance treatment or active surveillance-chemotherapy or other treatments. As many as 15-30% of patients with ovarian cancer have primary platinum-resistant or refractory disease. After recurrence, generally 70% of advanced stage ovarian cancer relapses, and even in stage I or II patients, the relapse rate is 20-25%. The recurrence of ovarian cancer after platinum-based chemotherapy is now considered a sign of an incurable disease. The average response rate to salvage therapy is only 10-15% with a median progression-free survival of 3-4 months. There is thus an unmet need for the development of new agents with activity against ovarian cancer, in particular against platinum-resistant ovarian cancer.

The inventors have discovered that their targeted delivery system comprising a CD45⁺ leukocyte cell comprising a complex of an iron binding protein and an active ingredient is capable of specifically targeting and killing ovarian cancer cells both in vitro and in an animal model of ovarian cancer. In addition, the treatment with the inventive targeted delivery system prevents metastatic spread in in an animal model of platinum-resistant ovarian cancer. Surprisingly, the treatment with the inventive targeted delivery system was more effective than the control paclitaxel treatment.

The inventive targeted delivery system for use in a method of treatment or diagnosis of ovarian cancer provides inter alia the following advantages over the prior art: (i) targeted delivery of active ingredients into ovarian cancer cells, (ii) improved penetration of active ingredients into deep sites of the ovarian cancer mass, (iii) improved distribution of active ingredients within the ovarian cancer mass, (iv) protection of active ingredients from inactivation in the blood circulation or clearance from the body, (v) delivery of active ingredients with poor pharmacokinetics into the ovarian cancer, (vi) reduced toxicity of active ingredients due to direct targeted delivery, (vii) higher treatment efficacy with lower doses of the active ingredient due to direct targeted delivery and local deposition in the ovarian cancer cell; (viii) improved efficacy of systemic administration of active ingredient, thereby preventing the risk of tissue injury during local delivery; (ix) efficient killing of tumor cells; (x) prevention of metastatic spread; (xi) improved treatment efficacy compared to standard therapies for ovarian cancer including paclitaxel treatment; and/or (xii) efficient treatment of platinum-resistant ovarian cancer.

### SUMMARY OF THE INVENTION

The present invention relates to an isolated targeted delivery system comprising a CD45⁺ leukocyte cell comprising within said cell a complex of one or more iron binding proteins and a pharmaceutically active substance, label or pharmaceutically active substance and label, for use in a method of treatment or diagnosis of ovarian cancer.

### DETAILED DESCRIPTION OF PREFERRED EMBODIMENTS

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

The terms “peptide” or “polypeptide” are used interchangeably in the context of the present invention to refer to a chain of at least two amino acids linked by peptide bonds. Thus, the term “polypeptide” in the context of the present invention is also used to refer to amino acid chains with more than 50, more than 100 or more than 150 amino acids.

The term “amino acid” encompasses naturally occurring amino acids as well as amino acid derivatives. In the context of the present specification, amino acids are identified using the 1-letter code (Hausman R E, Cooper G M (2004). The cell: a molecular approach. Washington, D.C. ASM Press. p. 51. ISBN 978-0-87893-214-6). An amino acid identified with the letter X corresponds to any amino acid. An amino acid identified with the letter B corresponds to either D (asparagine) or N (aspartic acid). An amino acid identified with the letter Z corresponds to either E (glutamine) or Q (glutamic acid).

The terms “polynucleotide” and “nucleic acid” are used interchangeably herein and are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA) and its various forms (e.g. but not limited to ssRNA, LNA etc.), deoxyribonucleic acid (DNA), and mixtures thereof such as e.g. RNA-DNA hybrids. The nucleic acids, can e.g. be synthesized chemically, e.g. in accordance with the phosphotriester method (see, for example, Uhlmann, E. & Peyman, A. (1990) Chemical Reviews, 90, 543-584). “Aptamers” are nucleic acids which bind with high affinity to a polypeptide. Aptamers can be isolated by selection methods such as SELEmir146-a (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability.

The term “identity” is used throughout the specification with regard to polypeptide and nucleotide sequence comparisons. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise. For example, a polypeptide sequence consisting of 200 amino acids compared to a reference 300 amino acid long polypeptide sequence may exhibit a maximum percentage of sequence identity of 66.6% (200/300) while a sequence with a length of 150 amino acids may exhibit a maximum percentage of sequence identity of 50% (150/300). If 15 out of those 150 amino acids are different from the respective amino acids of the 300 amino acid long reference sequence, the level of sequence identity decreases to 45%. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin&Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/or http://www.ebi.ac.uk/Tools/clustalw2/index.html. The grade of sequence identity (sequence matching) may be calculated using e.g. BLAST, BLAT or BlastZ (or BlastX). BLAST protein searches are performed with the BLASTP program, score=50, word length=3. To obtain gapped alignments for comparative purposes, Gapped BLAST is utilized as described in Altschul et al. (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl1: 154-162) or Markov random fields. Structure based alignments for multiple protein sequences and/or structures using information from sequence database searches, available homologs with 3D structures and user-defined constraints may also be used (Pei J, Grishin N V: PROMALS: towards accurate multiple sequence alignments of distantly related proteins. Bioinformatics 2007, 23:802-808; 3DCoffee@igs: a web server for combining sequences and structures into a multiple sequence alignment. Poirot O, Suhre K, Abergel C, O'Toole E, Notredame C. Nucleic Acids Res. 2004 Jul. 1; 32: W37-40.). When percentages of sequence identity are referred to in the present application, these percentages are calculated in relation to the full length of the longer sequence, if not specifically indicated otherwise.

The term “antibody” as used in the context of the present invention refers to a glycoprotein belonging to the immunoglobulin superfamily; the terms antibody and immunoglobulin are often used interchangeably. An antibody refers to a protein molecule produced by plasma cells and is used by the immune system to identify and neutralize foreign objects such as bacteria and viruses. The antibody recognizes a unique part of the foreign target, its antigen.

The term “antibody fragment” as used herein, refers to one or more fragments of an antibody that retain the ability to specifically bind to an antigen. Examples of binding fragments encompassed within the term “antibody fragment” include a fragment antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, an alternative scaffold protein, and a fusion protein thereof.

The term “antigen” is used to refer to a substance, preferably an immunogenic peptide that comprises at least one epitope, preferably an epitope that elicits a B or T cell response or B cell and T cell response.

An “epitope”, also known as antigenic determinant, is that part of a substance, e.g. an immunogenic polypeptide, which is recognized by the immune system. Preferably, this recognition is mediated by the binding of antibodies, B cells, or T cells to the epitope in question. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. The term “epitope” comprises both conformational and non-conformational epitopes. Conformational and non-conformational epitopes are distinguished in that the binding to the former but not the latter is lost in the presence of denaturing solvents.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

### Targeted Delivery System

The present invention relates to an isolated targeted delivery system comprising a CD45⁺ leukocyte cell comprising within said cell a complex of one or more iron binding proteins and a pharmaceutically active substance, label or pharmaceutically active substance and label, for use in a method of treatment or diagnosis of ovarian cancer.

The term “treatment” as used herein includes all types of preventive and/or therapeutic interventions medically allowed for the purpose of cure, temporary remission, prevention, etc. for different purposes including delaying or stopping the progress of a disease, making a lesion regress or disappear, preventing onset, or inhibiting recurrence. In instances where the isolated targeted delivery system is provided for use in a method of treatment of ovarian cancer, it comprises a pharmaceutically active substance. In instances where the isolated targeted delivery system is provided for use in a method of diagnosis of ovarian cancer, it comprises a label. The method of diagnosis is an in vivo method of diagnosis. In preferred embodiments, the isolated targeted delivery system is provided for use in a method of treatment of ovarian cancer. In some embodiments, the treatment comprises or consists of the killing of ovarian cancer cells. In some embodiments, the treatment comprises or consists of the prevention of metastatic spread.

The term “ovarian cancer” refers to cancers involving the ovaries, although they can arise from many different cell types within the ovaries: epithelial cells, germ cells, epidermoid cells, stromal cells, and border cells. Ovarian cancers of epithelial cell origin account for more than 85% of all ovarian tumors. Epithelial ovarian cancer is classified into five different histological subtypes: high-grade serous (HGS), low-grade serous (LGS), endometrioid, clear cell and mucinous. High-grade serous carcinomas are the most common ovarian cancer subtype (more than 70%) followed by endometrioid, clear cell and low-grade serous (Hirst, J., Crow, J., & Godwin, A. (2018). Ovarian Cancer Genetics: Subtypes and Risk Factors. In O. Devaja, & A. Papadopoulos (Eds.), Ovarian Cancer—From Pathogenesis to Treatment. IntechOpen. https://doi.org/10.5772/intechopen.72705). As many as 15-30% of patients with ovarian cancer have primary platinum-resistant or refractory disease. After recurrence, generally 70% of advanced stage ovarian cancer relapses, and even in stage I or II patients, the relapse rate is 20-25%. The recurrence of ovarian cancer is now considered a sign of an incurable disease (it becomes platinum-resistant). The average response rate to “salvage” therapy is only 10-15% with a median progression-free survival of 3-4 months.

In preferred embodiments of the invention, the ovarian cancer is an epithelial ovarian cancer, preferably and advanced epithelial ovarian cancer. In some embodiments, the epithelial ovarian cancer is selected from a low-grade serous (LGS) cell, endometrioid cell, clear cell and mucinous cell ovarian cancer. In some embodiments, the ovarian cancer is resistant to treatment with paclitaxel. In some embodiments, the ovarian cancer is resistant to or refractive after treatment with standard therapies for ovarian cancer, including chemotherapy with taxanes, chemotherapy with platinum (such as cisplatin, carboplatin), anti-VEGF monoclonal antibody therapy, and therapy with poly ADP-ribose polymerase inhibitors (PARPi). In some embodiments, the ovarian cancer is resistant to other therapies, in particular chemotherapies. In some embodiments, the chemotherapy is treatment with paclitaxel or platinum-based antineoplastic drugs, including, but not limited to, cisplatin, oxaliplatin, and carboplatin. In preferred embodiments, the ovarian cancer is platinum-resistant. Effectiveness in platinum-resistant ovarian cancer is especially important. There is a huge unmet need for an effective treatment especially for platinum-resistant patients. Even if ovarian cancer therapy pipeline is strong with several novel first-in-class molecules in late stage of clinical development, none of them is effective in platinum-resistant ovarian cancer treatment. In a another preferred embodiment, the ovarian cancer is resistant to treatment with paclitaxel.

In some embodiments, the isolated targeted delivery system is for use in a method of treatment of ovarian cancer in a human individual who experiences progressive disease or relapse during or following treatment with platinum-based antineoplastic drugs. In some embodiments, the isolated targeted delivery system is for use in a method of treatment of ovarian cancer in a human individual who experiences progressive disease or relapse during or following treatment with paclitaxel.

In the context of the present specification, the term “active ingredient” is used to refer to the at least one pharmaceutically active substance and/or at least one label. Preferably, the active ingredient is a pharmaceutically active substance.

The term “targeted delivery” refers to the delivery of a therapeutic or diagnostic agent (herein together referred to also as “active ingredient”) to a subject, e.g. patient, in particular to a cell, more particular directly into a cell within the body of a patient. The targeted delivery results in an increased concentration of the active ingredient in a particular region of the body when compared to administration of the active ingredient alone, administration of a complex of an iron binding protein and the active ingredient, or administration of other delivery systems. In particular, the targeted delivery results in an increased concentration of the active ingredient in tumor tissue, in particular ovarian cancer, more particularly inside ovarian cancer cells, when compared to administration of the active ingredient alone, administration of a complex of an iron binding protein and the active ingredient, or administration of other delivery systems. Targeted delivery also includes “targeted theragnostic delivery”, meaning that both a therapeutic and a diagnostic agent are delivered concomitantly, preferably to a diseased region, thus allowing simultaneous treatment and diagnosis and/or treatment monitoring.

In preferred embodiments, the active ingredient is delivered directly into the ovarian cancer cells, preferably via direct transfer from the CD45⁺ leukocyte to the ovarian cancer cell (direct cell-cell transfer). The direct transfer is preferably via a mechanism involving cell-cell contact and/or fusion of cell membranes.

The term “targeted pharmaceutically active substance delivery system” is used in the present application to refer to a system that is capable of delivering a pharmaceutically active substance to the targeted region, i.e. capable of targeted delivery within the body of a patient, preferably to a diseased region.

The term “targeted label delivery system” is used in the present application to refer to a system that is capable of delivering a label to the targeted region, i.e. capable of targeted delivery within the body of a patient, preferably to a diseased region.

The term “targeted theragnostic delivery system” is used in the present application to refer to a system that is capable of delivering a complex of a pharmaceutically active substance and at the same time a label to the targeted region, i.e. capable of targeted delivery within the body of a patient, preferably to a diseased region and thus allows simultaneous treatment and diagnosis and/or treatment monitoring.

The term “targeted delivery system” is used to commonly refer to “targeted pharmaceutically active substance delivery system”, “targeted label delivery system” and “targeted theragnostic delivery system”. Targeted delivery systems have been described in WO 2016/207257 A1, WO 2016/207256 A1 and WO 2017/222398 A1, which are incorporated herein by reference.

### Complex & Linker

Within the isolated targeted delivery system of the invention, the active ingredient may be covalently or non-covalently bound to the iron binding protein or may been capsulated by the iron binding protein or multimers thereof. The term “complex” also encompasses the enclosure of an active ingredient within an iron binding protein or multimers thereof, in particular multimers of ferritin forming a “ferritin cage”. In instance where the active ingredient is encapsulated within an iron binding protein or multimers thereof, the encapsulation may occur in the presence or absence of a covalent or non-covalent bond. In some embodiments, active ingredients can be encapsulated within the internal cavity of a ferritin oligomer (physical confinement) by exploiting the association/dissociation properties of the ferritin macromolecule itself. In such embodiments, the active ingredients are held in place by non-covalent interactions with amino acid residues within the cavity internal surface. Haemoglobin macromolecules also offer the possibility of non-covalent binding of selected pharmaceutically active substances and/or labels molecules that may be hosted within the heme binding pocket of haemoglobin itself. The heme in the pocket can be displaced and be replaced by pharmaceutically active substances and/or labels with appropriate hydrophobicity profile.

The formation of the complex allows the transport of the active ingredients into the CD45⁺ leukocyte when the CD45⁺ leukocyte is internalizing the iron binding protein. Thus, it is preferred that the active ingredients are bound to the iron binding protein in a way that does not interfere with the transport mechanism. This can be easily tested by the skilled person using uptake assays known in the art and described in WO 2016207257 A1, WO 2016207256 A1 and WO 2017222398 A1. If the complex comprising an active ingredient is taken up by a CD45⁺ leukocyte and transported to a target cell within the body, it is preferred that the complex is sufficiently stable to survive the transport within the cell to the target region within the body. Thus, it is preferred that the complex rather than the active ingredient alone is delivered to, preferably into the target cells in the target region. This property also reduces possible deleterious effects, e.g. cytotoxicity, of the active ingredient to the CD45⁺ leukocyte delivering the active ingredient or to other cells of the body that are not the target cells.

In preferred embodiments, the active ingredient and the iron binding protein are covalently and/or non-covalently linked, preferably covalently linked. If active ingredients are covalently linked to the iron binding proteins such coupling is preferably through amino acids residues known to be located in surface areas that are not involved in binding of the iron binding protein to a receptor involved in endocytosis.

In instances where the iron binding protein and the active ingredient are covalently linked, they may be linked directly or via a linker, i.e. indirectly. In preferred embodiments, the iron binding protein and the active ingredient are covalently linked via a linker.

Linkers are known to the skilled artisan, such as polyalanine, polyglycin, carbohydrates, (CH₂)ₙ groups or polypeptide linkers. The linker may be biodegradable or non-biodegradable, preferably biodegradable. In preferred embodiments, the linker is cleavable. The linker may be a peptide, disulfide, or hydrazone linker or a linker comprising carbohydrates. It is preferred that the linker is a peptide linker. In some embodiments, the peptide linker is cleavable by a protease. A linker comprising carbohydrates may be cleavable by β-glucuronidase. A hydrazone linker may be cleaved by acid hydrolysis. A disulfide linker may be cleaved by cytosolic reductive cleavage. In preferred embodiments, the linker, preferably a peptide linker, is cleavable by lysosomal proteases, more preferably lysosomal cysteine proteases, even more preferably cathepsins.

In some embodiments, the linker comprises a reactive group that, when activated by a defined stimulus, effects cleavage of a covalent bond within linker. Suitable reactive groups are e.g. light-activatable groups (i.e. groups that can be activated by UV light, such as 3-amino-3-(-2-nitro)phenyl-propionic acid or a light-activatable structural equivalent thereof), dithionite-activatable groups (such as an azobenzene moiety), or periodate-activatable groups (such as a 1,2-dihydroxy moiety, a 1-amino-2-hydroxy moiety or 4-amino-4-deoxy-L-threonic acid). In other embodiments, the linker is cleavable via a pH shift.

Pharmaceutically active substances or labels may also be covalently bound to iron binding protein amino acid side chains (lysines or cysteines) by appropriate choice of phenylhydrazone, succinimide or maleimide activated drugs. A phenylhydrazone derivative may break and liberate the drug from the iron binding protein, a lysine bound derivative may become active after full protein degradation into aminoacids, or a cysteine bound derivative may be liberated within the cell through reductive hydrolysis of the maleimede thioether link.

In some embodiments, the linker is a dipeptide linker, in particular Val-Cit, Val-Ala, or Ala-Ala, or a tripeptide and tetrapeptide linker.

In some embodiments, the linker is a hetero-bifunctional crosslinker that contains N-hydroxysuccinimide (NHS) ester and maleimide groups that allow covalent conjugation of amine- and sulfhydryl-containing molecules. In some embodiments, the linker is succinimidyl-4-(N-maleimidomethyl)cyclohexane-1-carboxylate (SMCC) or Sulfo-SMCC.

It is preferred that the cleavable linker is cleaved within the lysosomal compartment.

In most preferred embodiments, the linker is a maleimidocaproyl-valine-citrulline-para-aminobenzoyloxycarbonyl (mc-vc-PAB) linker.

In some embodiments, the iron binding protein is conjugated to the active ingredient or linker via a cysteine residue or a lysine residue, preferably a cysteine residue. For the formation of covalent bonds, relevant thiol, amino or carboxyl groups of the iron binding proteins are used to covalently couple active ingredients reactive towards thiol or amino groups directly or indirectly to the iron binding protein. The active ingredients may be modified by specific active moieties, i.e. linkers.

As such, ferritins or haemoglobin may be linked to cysteine thiol reactive pharmaceutically active substances and/or labels bearing a peptide based cleavable linker (e.g. cathepsin sensitive valine-citrulline sequence and para-aminobenzylcarbamate spacer). As a notable example, the antimitotic agent monomethyl auristatin E (MMAE) has been used. The peptide-based linker binds the protein to the cytotoxic compound in a stable manner so the drug is not easily released from the protein under physiologic conditions, which helps prevent toxicity to healthy cells and ensures dosage efficiency. The iron binding protein pharmaceutically active substance and/or label adduct thus generated is capable of attaching to the selected receptor types, e.g. CD163 for haemoglobin and TfR for transferrin, respectively. Once bound, the iron binding protein pharmaceutically active substance and/or label adduct is internalised by endocytosis and thus selectively taken up by the cells. The vesicle containing the active ingredient is fused with lysosomes and lysosomal cysteine proteases, particularly cathepsin B, start to break down the cleavable peptide linker, in particular the valine-citrulline linker, and the active ingredient, in particular MMAE, is no longer bound to the iron binding protein and is released directly into the tumour environment.

Alternatively, DM1-SMCC is an efficient mertansine derivative bearing a linker that specifically binds to lysine residues generating a covalent complex with ferritin, haemoglobin or transferrin in a reaction that has been successfully described for antibodies. In particular, haemoglobin, ferritin or transferrin can be reacted with DM1-SMCC thus providing a covalent protein-drug adduct that can be cleaved inside cells and releases the active drug in a time-dependent manner. The suppression of microtubule dynamics by DM1 induces mitotic arrest and cell death.

Ways of preparing complexes of an iron binding protein and an active ingredient are described in WO 2016/207257 A1, WO 2016/207256 A1 and WO 2017/222398 A1.

The term “full load” is used in the context of the present invention to refer to the maximum amount of iron binding protein, preferably ferritin, complexed with a pharmaceutically active substance, label or pharmaceutically active substance and label that can be taken up by the CD45⁺ leukocyte cell, preferably macrophage more preferably activated macrophage.

It is also envisioned that different active ingredients are comprised in the isolated targeted delivery system. For example, one type of active ingredient may be bound to a covalently bound to a ferritin polypeptide, while another type is encapsulated in the complex. This approach utilizes different release rates of the active ingredients from the complex once delivered to the targeted tissue and/or cells. For example, an active ingredient can be covalently attached to a ferritin molecule either on the surface of the 24-mer or within the internal cavity by exploiting the reactivity of relevant thiol, amino or carboxyl groups. The types of such useful reactions are well known in the art and can be adopted by the person skilled in the art to the particular active ingredient without any additional work. Examples of such reactions are described in Behrens C R, Liu B. Methods for site-specific drug conjugation to antibodies. MAbs. 2014 January-February; 6(1):46-53.

In theragnostic applications, i.e. in which the complex comprises both a label and a pharmaceutically active substance, it is preferred that the label is covalently attached to the iron binding protein and the pharmaceutically active substance is non-covalently bound to the iron binding protein and/or entrapped in the internal cavity formed upon assembly of the multimer of ferritin polypeptides.

### Iron Binding Protein

In some embodiments, the iron binding protein is selected from the group consisting of ferritin, preferably heavy (H) type ferritin, light (L) ferritin and/or mitochondrial ferritin; haemoglobin, preferably haemoglobin A, haemoglobin AS, haemoglobin SC, haemoglobin C, haemoglobin D, haemoglobin E, haemoglobin F, haemoglobin H; haemoglobin-haptoglobin complex, hemopexin, transferrin, and lactoferrin.

Human transferrin and ferritin proteins have been considered as effective carriers for the delivery of small molecules or toxin-conjugates to specifically target cancer cells. To date, in spite of considerable efforts, no successful transferrin or ferritin drug complexes have however reached the clinic (Luck A N et al. 2013, Adv Drug Deliv Rev 65(8):1012-9).

Ferritin is a hollow globular protein complex consisting of 24 ferritin monomer subunits assembled into a cage-like structure. Ferritin is the primary intracellular iron-storage protein. It is produced by almost all living organisms and is present in every cell type. Ferritin genes are highly conserved among species. In vertebrates, two ferritin monomers exist: the light (L) chain and the heavy (H) chain type with a molecular weight of 19 kDa or 21 kDa respectively. Vertebrate ferritin 24-mers can be homo-oligomers consisting of either L or H chains, or hetero-oligomers consisting of both L and H chains (Theil E C, 1987, Annual Review of Biochemistry. 56 (1): 289-315): Typically ferritin complexes have internal and external diameters of about 8 and 12 nm, respectively. Ferritin was shown to be internalized by endocytosis upon binding to CD71. Interaction of ferritin and CD71 is mediated via ferritin-H chains (Li L et al, Proc. Natl. Acad. Sci. USA 107 (8) (2010) 3505-3510). Ferritins are not abundant in plasma, but can be readily produced in high yield as recombinant proteins in common protein expression systems such as Escherichia coli cells.

Purified transferrin can be efficiently conjugated to various molecules including anticancer drugs through covalent linkers that are appropriately released inside the cells (Beyer U et al. 1998, J Med Chem 41 (15): 2701-2708). In case of transferrin, only lysine groups on the protein surface are ready available for covalent attachment.

Haemoglobin has been considered in the past as a possible drug carrier, due to its versatility in chemical conjugation with drugs, its abundance and relative stability in the blood (Somatogen, 1993, WO 1993/008842 A1). Nevertheless, the lack of receptor targeting properties did not foster biomedical applications other than blood substitutes or antisickling agent. As a matter of fact, Hb can only be recognized by CD163 (haptoglobin/haemoglobin receptor) epitopes on the leukocytes, especially monocyte-macrophage origin. The CD45⁺ leukocyte, in particular macrophage based protein delivery, described in this application moved haemoglobin center stage as a target specific carrier of pharmaceutically active substances and/or labels. Haemoglobin can be readily covalently linked to appropriate pharmaceutically active substances and/or labels, host hydrophobic pharmaceutically active substances and/or labels within the heme binding pocket or even transport small molecules, e.g. cytotoxic molecules linked to the heme iron. Hb can be easily modified by selective attachment of the appropriate drug conjugate to the beta93 cysteine residue, the only titratable cysteine on the protein surface. Maleimido functionalized drugs, such as the tubulin inhibitor Monomethyl auristatin (MMAE) or the DNA crosslinking drug Pyrrolobenzodiazepine dimer (PBD) are most notable examples of extremely potent cytotoxic agents that can be readily and specifically attached to the relevant cys beta93 residue.

Alternatively, lysine residues on the Hb surface (at least 10 titratable lysine residues per Hb tetramer) may be easily amenable to drug conjugation through cleavable succinimide linkers. Haemoglobin also offers a unique capability of releasing non covalently bound heme group at acidic pH values. Apo-haemoglobin thus obtained is capable of hosting several hydrophobic molecules within the empty heme pocket, as shown in the case of paclitaxel (Meng Z et al. 2015 J Pharm Sci 104(3):1045-55) or for labels with fluorescent properties (e.g. chlorine e6, hyperycin, phtalocyanine derivatives) (Dong J et al. J PhotochemPhotobiol B 2014, 140:163-172).

In a preferred embodiment of the targeted delivery system for use of the present invention, the iron binding protein is selected from the group consisting of ferritin, preferably heavy (H) type ferritin, light (L) ferritin and/or mitochondrial ferritin; haemoglobin, preferably haemoglobin A, haemoglobin AS, haemoglobin SC, haemoglobin C, haemoglobin D, haemoglobin E, haemoglobin F, haemoglobin H; haemoglobin-haptoglobin complex, haemopexin, transferrin; and lactoferrin. The terms ferritin; haemoglobin, preferably haemoglobin A, haemoglobin AS, haemoglobin SC, haemoglobin C, haemoglobin D, haemoglobin E, haemoglobin F, haemoglobin H; haemoglobin-haptoglobin complex, hemopexin, transferrin; and lactoferrin encompass structural variants of the naturally occurring proteins and, thus relates to proteins that have at least 70%, preferably at least 75%, more preferably at least 80%, more preferably at least 85%, more preferably at least 90%, more preferably at least 95% more preferably at least 100% of the ability of the respective wild-type protein to bind iron ion(s). The iron binding proteins used in the context of the present invention are preferably of mammalian, more preferably mouse, rat, dog, ape, in particular, chimpanzee, or human, most preferably of human origin. Consensus sequences of the preferred iron binding proteins and preferred structural variants are disclosed in WO 2016/207257 A1.

In a preferred embodiment, the iron binding protein is ferritin, preferably a mammalian ferritin. The mammalian ferritin may be a mouse, rat, dog, ape, in particular chimpanzee, or human ferritin. In a preferred embodiment, the mammalian ferritin is a mouse, rabbit, rat or human ferritin, preferably human ferritin. In an even more preferred embodiment, the human ferritin is a human heavy chain ferritin.

Amino acid substitutions within the iron binding proteins are preferably selected in a way that they do not unduly change the conformation of the polypeptide. As an example, a “small amino acid” should be substituted with another small amino acid. A “small amino acid” in the context of the present invention is preferably an amino acid having a molecular weight of less than 125 Dalton. Preferably, a small amino acid in the context of the present invention is selected from the group consisting of the amino acids glycine, alanine, serine, cysteine, threonine, and valine, or derivatives thereof. As another example, an amino acid having a hydrophobic side chain should be substituted with another amino acid having a hydrophobic side chain.

In some embodiments, the ferritin comprises an amino acid sequence having at least 70%, at least 80%, at least 85%, at least 90%, at least 95%, at least 98%, at least 99% or 100% identity to any one of SEQ ID NO: 1-5 and has at least 70%, preferably at least 75%, more preferably at least 80%, more preferably at least 85%, more preferably at least 90%, more preferably at least 95% of the ability of wild-type ferritin, in particular human wild type ferritin, to bind iron ion(s) and/or form ferritin 24-mers. SEQ ID NO: 3 is a mammalian consensus sequence. In SEQ ID NO: 3, X at position 6 can be any naturally occurring amino acid, preferably Pro X at position 14 can be any naturally occurring amino acid, preferably His, X at position 16 can be any naturally occurring amino acid, preferably Asp, X at position 21 may be present or absent, if present it means any amino acid, preferably Ile, X at position 29 can be any naturally occurring amino acid, preferably Tyr, X at position 81 can be any naturally occurring amino acid, preferably Phe, X at position 83 can be any naturally occurring amino acid, preferably Gln, X at position 105 can be any naturally occurring amino acid, preferably His, X at position 144 can be any naturally occurring amino acid, preferably Ala or Ser, more preferably Ala, X at position 180 is absent or any naturally occurring amino acid, preferably Asn, X at position 181 is absent or any naturally occurring amino acid, preferably Glu, X at position 182 is absent or any naturally occurring amino acid, preferably Ser. SEQ ID NO: 5 is a mammalian consensus sequence. In SEQ ID NO: 5, X at position 6 can be any naturally occurring amino acid, preferably Pro, X at position 14 can be any naturally occurring amino acid, preferably His, X at position 16 can be any naturally occurring amino acid, preferably Asp, X at position 21 may be present or absent, if present it means any amino acid, preferably Ile, X at position 22 means any amino acid, preferably Asn, X at position 30 can be any naturally occurring amino acid, preferably Tyr, X at position 40 can be any naturally occurring amino acid, preferably Tyr or Cys, more preferably Tyr, X at position 82 can be any naturally occurring amino acid, preferably Phe, X at position 84 can be any naturally occurring amino acid, preferably Gln, X at position 91 can be any naturally occurring amino acid, preferably Arg or Cys, more preferably Cys, X at position 106 can be any naturally occurring amino acid, preferably His, X at position 110 can be any naturally occurring amino acid, preferably Asn or Ser, more preferably Asn, X at position 137 can be any naturally occurring amino acid, preferably His or Tyr, more preferably His, X at position 140 can be any naturally occurring amino acid, preferably Asn or Ser, more preferably Asn, X at position 145 can be any naturally occurring amino acid, preferably Ala or Ser, more preferably Ala, X at position 164 can be any naturally occurring amino acid, preferably Ala or Ser, more preferably Ser, X at position 166 can be any naturally occurring amino acid, preferably Met or Leu, preferably Leu, X at position 178 can be any naturally occurring amino acid, preferably Asp or His, more preferably Asp, X at position 181 is absent or any naturally occurring amino acid, preferably Asn, X at position 182 is absent or any naturally occurring amino acid, preferably Glu, X at position 183 is absent or any naturally occurring amino acid, preferably Ser. In some embodiments, the ferritin comprises an amino acid sequence according to SEQ ID NO: 4, optionally comprising 1-5, 1-10, 1-15, 1-20 or 1-25 amino acid mutations outside position 54, 72, 87 and/or 144, in particular outside position 54, and having at least 70%, preferably at least 75%, more preferably at least 80%, more preferably at least 85%, more preferably at least 90%, more preferably at least 95% of the ability of wild-type ferritin, in particular human wild type ferritin, to bind iron ion(s) and/or form ferritin 24-mers, in particular to form ferritin 24-mers.

Any ferritin used in the isolated targeted delivery system for use according to the invention has to retain the properties of a wild type ferritin with regard to complex formation (cage-like structure consisting of 24 ferritin monomer subunits) and uptake of iron.

### Drugs and Labels

The terms “drug” or “pharmaceutically active substance” are used synonymously in the context of the present invention and refer to any compound that modifies or modulates cell activity or is capable of being activated, i.e. a prodrug, to modify or modulate cell activity, preferably in the body of a patient. Examples of such active ingredients include so called “small molecules” and peptides. The term “small molecule” is used in the context of the present invention to refer to a hydrocarbon with a molecular mass of below 1.500 g/mol or to pharmaceutically active radioactive isotopes.

In preferred embodiments, the pharmaceutically active substance is an anticancer drug selected from the group consisting of a protein, a peptide, a nucleic acid, a non-protein non-nucleic acid compound with a molecular weight of less than 1.5 kD, a photosensitizing compound, a virus, and pharmaceutically active radioactive isotope.

A preferred pharmaceutically active radioactive isotope is an a or β radiation emitting radioisotope, which also emits a cell damaging amount of γ radiation is selected from the group consisting of lutetium-177, ytterbium-90, iodine-131, samarium-153, phosphorus-32, caesium-131, palladium-103, radium-233, iodine-125, and boron-10 or a cell damaging amount of a radiation, preferably selected from the group consisting of actinium-225, bismuth-213, lead-212, and polonium-212. Also preferred is a complex of above mentioned compounds and isotopes linked to the nanoparticles (e.g. gold, argentum, graphen) or these nanoparticles.

If the pharmaceutically active substance is a virus, it is preferably an oncolytic virus.

If the drug is a nucleic acid it is preferred that it is a miRNA, siRNA, chemically modified-RNA, LNA, ssRNA, DNAzyme or a nucleic acid encoding a pharmaceutically active protein, e.g. an antibody, an antibody mimetic, a cytokine, a prodrug-converting enzyme, an immunogenic peptide or the like.

In a preferred embodiment, the anticancer drug is a cytostatic drug, cytotoxic drug or prodrug thereof.

Preferred anticancer drugs are selected from an apoptosis/autophagy or necrosis-inducing drug. An apoptosis/autophagy or necrosis-inducing drug can be any drug that is able to induce apoptosis/autophagy or necrosis effectively even in cells having an abnormality in cell proliferation. These drugs are preferably used in complexes with one or more ferritins.

In preferred embodiments, the anti-cancer drug is selected from the group consisting of an apoptosis-inducing drug, an alkylating substance, anti-metabolites, antibiotics, antimitotic agent, a DNA-modifying drug, a DNA minor groove interstrand crosslinking drug, an inhibitor of DNA synthesis, an inhibitor of RNA synthesis, epothilones, nuclear receptor agonists and antagonists, an anti-androgene, an anti-estrogen, a platinum compound, a hormone, a antihormone, an interferon, an inhibitor of cell cycle-dependent protein kinases (CDKs), an inhibitor of cyclooxygenases and/or lipoxygenases, a biogenic fatty acid, a biogenic fatty acid derivative, including prostanoids and leukotrienes, an inhibitor of protein kinases, an inhibitor of protein phosphatases, an inhibitor of lipid kinases, a platinum coordination complex, an ethyleneimine, a methylmelamine, a triazine, a vinca alkaloid, a pyrimidine analog, a purine analog, an alkylsulfonate, a folic acid analog, an anthracendione, a substituted urea, and a methylhydrazin derivative, an ene-diyne antibiotic, a maytansinoid, an auristatin derivate, an immune check-point inhibitor, and an inhibitor of a tumour-specific protein or marker, preferably a Rho-GDP-dissociation inhibitor, more preferably Grp94 or AXL inhibitor, a tubulin inhibitor, or a topoisomerase inhibitor.

In preferred embodiments, the anti-cancer drug is selected from the group consisting of acediasulfone, aclarubicine, ambazone, aminoglutethimide, L-asparaginase, auristatin, azathioprine, banoxantrone, bendamustine, bleomycin, busulfan, calcium folinate, carboplatin, carpecitabine, carmustine, celecoxib, chaliceamycin, chlorambucil, cis-platin, cladribine, cyclophosphamide, cytarabine, dacarbazine, dactinomycindapsone, daunorubicin, deruxtecan, dibrompropamidine, diethylstilbestrole, docetaxel, doxorubicin, dolastatin 10, dolastatin 15, dynemycinA, enediynes, epirubicin, epothilone B, epothilone D, estramucin phosphate, estrogen, ethinylestradiole, etoposide, exatecane derivative, flavopiridol, floxuridine, fludarabine, fluorouracil, fluoxymesterone, flutamidefosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon α, irinotecan, leuprolide, lomustine, lurtotecan, mafenide sulfate olamide, maytansine, mechlorethamine, medroxyprogesterone acetate, megastrolacetate, melphalan, mepacrine, mercaptopurine, mertansine, methotrexate, metronidazole, mitomycin C, mitopodozide, mitotane, mitoxantrone, mithramycin, nalidixic acid, neocazinostatin, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, pyrrolobenzodiazepine, raltitrexed, rapamycin, rofecoxib, rosiglitazone, salazosulfapyridine, scriflavinium chloride, semustinestreptozocine, sn-38, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, tinidazole, topotecan, triaziquone, treosulfan, trimethoprim, trofosfamide, UCN-01, vinblastine, vincristine, vindesine, vinblastine, vinorelbine, and zorubicin. More preferably, the anti-cancer drug is selected from the group consisting of auristatin, banoxantrone, bendamustine, chlorambucil, chaliceamycin, dynemycin A, maytansine, melphalan, mertansine, neocazinostatin and pyrrolobenzodiazepine.

In particularly preferred embodiments, the anti-cancer drug is auristatin, in particular monomethyl auristatin E or monomethyl auristatin F. In particularly preferred embodiments, the anti-cancer drug is deruxtecan. The examples were performed using the drug monomethyl auristatin E.

In some embodiments, the anti-cancer drug is an immunomodulatory drug that activates or inhibits an activity of an immune cell, preferably the immunomodulatory drug is a ligand or antagonist of Pattern Recognition Receptors, particularly Toll-like Receptors, NOD-like receptors (NLR), RIG-I-like receptors (RLR) or Stimulator of interferon genes (STING) protein. Physiologically, these receptors recognize classes of signals known as pathogen-associated molecular patterns (PAMPs) and damage-associated molecular patterns (DAMPs).

In preferred embodiments, the anti-cancer drug is a proliferation inhibiting protein or peptide, preferably a cell cycle inhibitor or an antibody or antibody like binding protein that specifically binds to a proliferation promoting protein or a nucleic acid, preferably encoding a proliferation inhibiting protein or an antibody or antibody like binding protein that specifically binds to a proliferation promoting protein or a siRNA, oligonucleotide, LNA, or DNAzyme.

The term “prodrug” as used in the context of the present invention refers to any active ingredient that, after administration, is metabolized or otherwise converted to a biologically active or more active ingredient (or drug) with respect to at least one property. In comparison to the drug, a prodrug is modified chemically in a manner that makes it, relative to the drug, less active or inactive, but the chemical modification is such that the corresponding drug is generated by metabolic or other biological processes after the prodrug is administered to the patient. A prodrug may for example have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

In some embodiments, the pharmaceutically active substance is a hypoxia-activated prodrug, preferably selected from the group consisting of benzotriazine N-oxides, apaziquone (E09), tirapazamine (TPN), SN30000, PR-104A, TH-302, TH-4000 and AQ4N. The use of active ingredients, which are activated under hypoxic conditions can add a further specificity to the targeting and/or further reduces adverse effects of the active ingredients. Thus, in particularly preferred embodiments the active ingredient is a hypoxia-activated prodrug. The backbone of all the hypoxia-activated prodrugs is the presence of one of five different chemical moieties (nitro groups, quinines, aromatic and aliphatic N-oxides and transition metals) that are enzymatically reduced under hypoxic conditions in tissue. Hypoxia-activated prodrugs are any prodrug that is less active or inactive, relative to the corresponding drug, and comprises the drug and one or more bioreducible groups. Such hypoxia-activated prodrugs include all prodrugs activated by a variety of reducing agents and reducing enzymes, including without limitation single electron transferring enzymes (such as cytochrome P450 reductases) and two electron transferring (or hydride transferring) enzymes. According to preferred embodiment of the invention hypoxia-activated prodrug is TH-302. Methods of synthesizing TH-302 are described in PCT application WO 07/002931 and WO 08/083101.

In some embodiments, the pharmaceutically active substance is an antigen or a nucleic acid encoding an antigen.

The terms “label” or “diagnostic agent” are used interchangeably herein and refer to any kind of compound being suitable for diagnostic purposes. In a preferred embodiment, the label is selected from the group consisting of a fluorescent dye, a radioisotope/fluorescence emitting isotope, a detectable polypeptide or nucleic acid encoding a detectable polypeptide, and a contrast agent or the label comprises a chelating agent which forms a complex with divalent or trivalent metal cations. More preferably, the label is selected from a fluorescent dye, a radioisotope and a contrast agent. A contrast agent is a dye or other substance that helps to show abnormal areas inside the body.

Preferred fluorescent dyes are selected from the group consisting of the following classes of fluorescent dyes: xanthens (e.g. fluorescein), acridines (e.g. acridine yellow), oxazines (e.g. oxazine 1), cynines (e.g. Cy7/Cy 3), styryl dyes (e.g. dye-28), coumarines (e.g. Alexa Fluor 350), porphines (e.g. chlorophyll B), metal-ligand-complexes (e.g. PtOEPK), fluorescent proteins (e.g. APC, R-phycoerythrin), nanocrystals (e.g. QuantumDot 705), perylenes (e.g. Lumogen red F300) and phtalocyanines (e.g. IRDYE™700DX) as well as conjugates and combinations of these classes of dyes.

Preferred radioisotopes/fluorescence emitting isotopes are selected from the group consisting of alpha radiation emitting isotopes, gamma radiation emitting isotopes, Auger electron emitting isotopes, X-ray emitting isotopes, fluorescent isotopes, such as 65Tb, fluorescence emitting isotopes, such as 18F, 51Cr, 67Ga, 68Ga, 89Zr, 111In, 99mTc, 140La, 175Yb, 153Sm, 166Ho, 88Y, 90Y, 149Pm, 177Lu, 47Sc, 142Pr, 159Gd, 212Bi, 72As, 72Se, 97Ru, 109Pd, 105Rh, 101m15Rh, 119Sb, 128Ba, 123I, 124I, 131I, 197Hg, 211At, 169Eu, 203Pb, 212Pb, 64Cu, 67Cu, 188Re, 186Re, 198Au and 199Ag as well as conjugates and combinations of above with proteins, peptides, small molecular inhibitors, antibodies or other compounds (e.g. 18F-FDG, 89Zr-oxide or 64Cu-porfirin).

Preferred detectable polypeptides are an autofluorescent protein, preferably green fluorescent protein or any structural variant thereof with an altered adsorption and/or emission spectrum.

Preferred contrast agents are selected from paramagnetic agents, e.g. Gd, Eu, W and Mn, preferably complexed with a chelating agent. Further options are superparamagnetic iron (Fe) complexes and particles, compounds containing atoms of high atomic number, i.e. iodine for computer tomography (CT), microbubbles and carriers such as liposomes that contain these contrast agents. In a preferred embodiment, the label comprises a chelating agent which forms a complex with divalent or trivalent metal cations.

Preferred chelating agents are selected from the group consisting of 1,4,7,10-tetraazacyclododecane-N,N′,N,N′-tetraacetic acid (DOTA), ethylenediaminetetraacetic acid (EDTA), 1,4,7-triazacyclononane-1,4,7-triacetic acid (NOTA), triethylenetetramine (TETA), iminodiacetic acid, Diethylenetriamine-N,N,N′,N′,N″-pentaacetic acid (DTPA) and 6-Hydrazinopyridine-3-carboxylic acid (HYNIC).

### Leukocyte

The ability of a given cell or of a population thereof to internalize ferritin depends on the expression of receptors involved in this internalization process. Receptors that lead to internalization of ferritin comprise, e.g. TfR, CXCR4, scavenger receptors, CD163, and TIM-2. The skilled person is well aware how to measure the amount of ferritin uptake and preferred methods of measuring the uptake are described in the Example Section below.

The term “leukocyte” (or “leukocyte cell”) is used in the context of the present invention to refer to cells of the immune system that are involved in protecting the body against both infectious disease and foreign invaders. All leukocytes are produced and derived from multipotent cells in the bone marrow known as a hematopoietic stem cells. Leukocytes are found throughout the body, including the blood and lymphatic system. All leukocytes have nuclei, which distinguishes them from the other blood cells, the anucleated red blood cells (RBCs) and platelets. Types of leukocyte can be classified in standard ways. Two pairs of the broadest categories classify them either by structure (granulocytes or agranulocytes) or by cell division lineage (myeloid cells or lymphoid cells). These broadest categories can be further divided into the five main types: neutrophils, eosinophils, basophils, lymphocytes, and monocytes. These types are distinguished by their physical and functional characteristics. Monocytes and neutrophils are phagocytic. Further subtypes can be classified; for example, among lymphocytes, there are B cells, T cells, and NK cells. Granulocytes are distinguished from agranulocytes by their nucleus shape (lobed versus round, that is, polymorphonuclear versus mononuclear) and by their cytoplasm granules (present or absent, or more precisely, visible on light microscopy or not thus visible). The other dichotomy is by lineage: Myeloid cells (neutrophils, monocytes, eosinophils and basophils) are distinguished from lymphoid cells (lymphocytes) by hematopoietic lineage (cellular differentiation lineage).

CD45 expression is characteristic of a subgroup of leukocyte cells, i.e. monocyte, monocyte-macrophages, lymphocytes, granulocytes, NK cells that are suitable to be used in the context of the targeted delivery system of the present invention, in particular since CD45⁺ leukocyte cells are attracted to particular tissues and cells within the body and are capable of delivering complexes of one or more iron binding proteins and one or more pharmaceutically active substances, labels or pharmaceutically active substances and labels to or into cells. This subgroup of leukocytes is in the following referred to as “CD45⁺ leukocyte cells” or “CD45⁺ leukocytes”. Preferably the monocyte is not a dendritic cell which differentiation is controlled by one or more of the following transcription factors: IFN-regulatory factor 8 (IRF8), nuclear factor interleukin (IL)-3-regulated protein (NFIL3), basic leucine zipper transcriptional factor ATF-like 3 (BATF3) or Transcription Factor RelB (NF-KB Subunit)—RELB, Spi-1 Proto-Oncogene (PU/1), recombining binding protein suppressor of hairless (RBPJ), IFN-regulatory factor 4 (IRF4) or transcription factor E2-2 (also known as (TCF4).

It is understood by the skilled person that CD45⁺ leukocyte cells as defined above unless of clonal origin are a mixed population of different leukocytes which share the common property of expressing CD45 surface antigen. Accordingly, subpopulations of cells within the diverse group of CD45⁺ leukocyte cells as defined above are characterized throughout the specification by further functional and/or structural characteristics. The term “CD45⁺” indicates that the majority of cells within a population of cells or essentially all cells express the CD45 surface antigen.

“Expressing” means in this respect that the majority of cells within a population of cells or essentially all cells express the marker (also called surface antigen herein). In this context and also with reference to other cellular surface antigens, the term “expresses” indicates that the surface antigen is produced within the cell and detectably exposed on the surface of a cell. The level of expression and, thus the number of surface antigens detectably exposed on the surface of a cell can vary greatly among different cells. Generally, a cell is considered to be positive, i.e. is indicated to be “⁺”, for a cellular surface antigen, if at least 5, preferably at least 10 copies of the surface antigen are detectably exposed on the surface of the cell. The skilled person is well aware of how to detect, quantify and select for cells, which are positive (or negative) for a given cellular surface antigen. Preferred methods include Fluorescence Activated Cell Sorting (FACS). In this technology fluorescently labelled antibodies are used to bind to cellular surface antigens of a population of cells, the cells are subsequently isolated into single cells and based on fluorescence intensity measured for the single cell, characterized as being positive or negative for the given cellular surface antigen. In some embodiments of the present invention it is indicated that the expression of a given protein is high or low. This means that the protein is detectably expressed in both instances, i.e. is “⁺”, however, at different levels. High and low expression, respectively, will mean different absolute numbers of proteins per cell for different proteins. Thus, a given protein may be considered to be expressed at high levels if there are more than 500 detectable copies of that protein per cell and to be expressed at low levels if there are between 1 to 50 detectable copies of that protein per cell. However, another protein may be considered to be expressed at high levels, if there are more than 5000 detectable copies and expressed at low levels, if there are between 1 to 500 detectable copies per cell. It is well known in the art how to quantify the number of proteins expressed or produced in a cell using flow cytometry and Becton Dickinson Quantibrite™ bead method (see e.g. Pannu, K. K., 2001, Cytometry. 2001 Dec. 1; 45 (4): 250-8) or mass spectrometry (see, e.g. Milo, R., 2013, Bioessays, 35 (12): 1050-1055).

For the purpose of the present invention the term “high expression” of a given protein refers to detectable expression of that protein that is at least 70% of the highest expression level found, i.e. number of copies per cell, in a population of healthy cells, in particular CD45⁺ leukocytes. The term “low expression” of a given protein refers to detectable expression of that protein that is 30% or less of the highest expression level found, i.e. number of copies of that protein per cell, in a population of healthy cells, in particular CD45⁺ leukocytes. Preferably, the “highest expression level” is determined as the average of the highest expression levels found in healthy cells, in particular CD45⁺ leukocytes of different subjects. In some embodiments preferred subpopulations of cells are characterized as “producing” a given protein. This is understood to mean that the protein is not necessarily detectable on the surface of the cell but may only be present inside the cell. The skilled person is well aware how to detect and/or quantify production of a protein inside a cell and/or select cells producing such proteins. Alternatively, cell populations can be defined by expression of specific transcription factors. It is well known in the art how to determine expression of a given protein or its encoding mRNA in a population of cells or even in single cells, e.g. using in vivo labeling with antibodies, FISH assays, in vivo single molecule fluorescent microscopy (Crawford, R. et al. Biophys J. (2013) 105 (11): 2439) alone or in combination with Fluorescent Activated Cell Sorting (FACS), or by the PrimeFlow technique (e Bioscience), (Adam S. Venable, et. al., (2015) Methods in Molecular Biology).

The term “differentiated monocyte” is used in the context of the present invention to refer to a monocyte differentiated from the committed precursor termed macrophage-DC precursor (MDP) mainly resident in bone marrow (but could be also in the spleen) and differentiate into either dendritic cells or macrophages. In mice they consist of two main subpopulations: (i) CD11b⁺ cell with high expression of CX3CR1, low expression of CCR2 and Ly6C- and (ii) CD11b⁺ cell with low expression of CX3CR1, high expression of CCR2 and Ly6C⁺. After leaving the bone marrow, mouse Ly6C⁺ monocytes differentiate into Ly6C-monocytes in circulation. Similarly, in human monocyte differentiation, it is accepted that CD14⁺⁺ classical monocytes leave bone marrow and differentiate into CD14⁺⁺CD16⁺ intermediate monocytes and sequentially to CD14⁺CD16⁺⁺ non-classical monocytes in peripheral blood circulation (Yang et al. 2014; Biomark Res 2 (1) doi. 10.1186/2050-7771-2-1). Preferably the differentiated monocyte is not a dendritic cell, which differentiation is controlled by one or more of the following transcription factors: IRF8, NFIL3, BATF3, RELB, PU/1, RBPJ, IIRF4, and/or TCF4, and more preferably is not a dendritic cell.

Macrophages are tissue-resident professional phagocytes and antigen-presenting cells (APC), which differentiate from circulating peripheral blood monocytes (PBMs). The term “activated macrophage” is used in the context of the present invention to refer to any macrophage that is polarized. Unpolarized macrophages are referred to as M0 macrophages herein. Macrophage activation is in general achieved by incubation with interleukins, cytokines and/or growth factors. In particular IL-4 and M-CSF can be used as activating agents. Activated macrophages of different phenotypes are classified into M1-macrophages, classically activated macrophages (CAM) and M2-macrophages, alternatively activated macrophages (AAM). The classically activated M1-macrophages comprise immune effector cells with an acute inflammatory phenotype. These are highly aggressive against bacteria and produce large amounts of lymphokines (Murray, and Wynn, 2011, J LeukocBiol, 89 (4): 557-63). The alternatively activated, anti-inflammatory M2-macrophages can be separated into at least three subgroups. These subtypes have various different functions, including regulation of immunity, maintenance of tolerance and tissue repair/wound healing. The term “M1 inducer” is used in the context of the present invention to refer to a compound that directs differentiation of PBMs to macrophages of the M1 type. The term “M2 inducer” is used in the context of the present invention to refer to a compound that directs differentiation of PBMs to macrophages of the M2 type. The skilled person is aware of a large number of ways to promote differentiation into either M1 or M2 macrophages. The term “phagocytosis by macrophages” is the process by which a macrophage engulfs a solid particle to form an internal vesicle known as a phagosome. The expressions “viral/bacterial/fungal/helminth proteins or products” refers to molecules produced by or originating from viruses, bacteria, fungi or helminths during a viral/bacterial/fungal/helminth infection.

The CD45⁺ leukocyte cell comprised in the targeted delivery system for use according to the invention is producible from a CD34⁺ hematopoietic precursor cell.

In preferred embodiments, the CD45⁺ leukocyte is selected from the group consisting of a monocyte, a differentiated monocyte, preferably a macrophage, a lymphocyte and a granulocyte. It is preferred that the CD45⁺ leukocyte is a macrophage, preferably an activated macrophage.

With respect to the monocyte, it is preferred that it is a CD11b⁺ monocyte, preferably selected from the group consisting of a CD11b⁺CD14⁺ monocyte, a CD11b⁺CD16⁺ monocyte, a CD11b⁺CD14⁺ CD16⁺ monocyte, a CD11b⁺CD14 HLA-DR monocyte, a CD11b⁺CD14⁺CD115⁺ monocyte, a CD11b⁺CD14⁺ monocyte, a CD11b⁺CD16⁺ monocyte, a CD11b⁺CCR1⁺ monocyte, a CD11b⁺CCR2⁺ monocyte, a CD11b⁺CX3CR⁺ monocyte, a CD11b⁺CXR4⁺ monocyte, a CD11b⁺CXR6⁺ monocyte and a CD11b⁺CD14⁺CD33⁺ monocyte.

With respect to the differentiated monocyte, it is preferred that it is selected from the group consisting of a macrophage, an activated macrophage, preferably a CD11b⁺ macrophage, more preferably a CD11b⁺CD16⁺ macrophage, a CD11b⁺CD32⁺ macrophage, a CD11b⁺CD64⁺ macrophage, a CD11b⁺CD68⁺ macrophage, preferably a CD11b⁺CD86+M1 macrophage, preferably producing iNOS and/or secreting interleukin 12 (IL-12) or preferably a CD11b⁺CCR2+M2 macrophage, a CD11b⁺CD204⁺ M2 macrophage, a CD11b⁺CD206⁺ M2 macrophage, a CD11b⁺CD204⁺CD206+M2 macrophage, a CD11b⁺HLA-DR⁺ M2 macrophage, a CD11b⁺CD200R⁺M2 macrophage, a CD11b⁺CD163⁺ M2 macrophage or an activated macrophage producing arginase and/or secreting interleukin 10 (IL-10); and a dendritic cell (DC), preferably a CD11b⁺CD11c⁺DC, CD11b⁺CD80⁺DC, CD11c⁺CD80⁺DC, CD11c⁺CD86⁺DC, CD11c⁺HLA-DR⁺DC or CD11c⁺CD123 DC, preferably the differentiated monocyte-macrophage is not a Lox1⁺, CXCR7⁺ and NRF2⁺ foam cell.

In some embodiments, the macrophage is a differentiated macrophage, in particular an M1 macrophage or an M2 macrophage. In some embodiments, the macrophage is an undifferentiated macrophage. In some embodiments, the macrophage is a naive macrophage. In some embodiments, the macrophage is an unpolarized macrophage. In preferred embodiments, the macrophage is an M0 macrophage or a macrophage that is mildly polarized towards M2. In some embodiments, the macrophage is an M2 macrophage. The skilled person is aware of surface markers expressed by M2 macrophages or macrophages that are mildly polarized towards M2.

Preferably, the differentiated monocyte expresses at least one chemokine receptor, preferably selected from the group consisting of CCR1, CCR2, CXCR4, and CXCR6, or at least one growth factor receptor, preferably selected from the group consisting of macrophage colony-stimulating factor receptor (CD115), granulocyte colony-stimulating factor receptor (CD114), and granulocyte-macrophage colony stimulating factor receptor (CD116 and CD131).

In preferred embodiments, the monocyte or differentiated monocyte:
(i) is producible from a CD34⁺ hematopoietic precursor cell;
(ii) is producible by in vitro incubation of monocytes with at least one inducer, preferably M1 or M2 inducer, more preferably at least one M2 inducer;
(iii) is characterized by expression of at least one of the following antigens: TfR, CD163, CD14, CD16, CD33, CXCR4, 25f9, HLA-DR and/or CD115 and optionally CD172a and/or CXCR4, in particular at least one of TfR, CD163, CD14, CD16, CD33, 25f9, CD172a and/or CD115 or at least one of TfR, CD163, CD14, CD16, CXCR4, 25f9, and/or CXCR1; and/or
(iv) has the ability to phagocytose.

Preferably,
(i) the M1 inducer is selected from the group consisting of LPS, GM-CSF, INF-γ, viral or bacterial proteins or products;
(ii) the M2 inducer is selected from the group consisting of IL-4, IL-10, IL-13, an immune complex of an antigen and antibody, IgG, heat activated gamma-globulins, glucocorticosteroids, TGF-β, IL-1R, CCL-2, IL-6, M-CSF, PPARγ agonist, leukocyte inhibitory factor, cancer-conditioned medium, cancer cells, adenosine and helminth or fungal proteins or products.

In preferred embodiments, the activated macrophage:
(i) is producible by in vitro incubation of a monocyte or macrophage with a factor capable of altering expression markers on macrophages, preferably
   (a) with at least one M1 inducer,
   (b) with at least one M2 inducer,
   (c) or with a factor capable of altering the macrophages' ability to secrete cytokines, preferably IL-10 and IL-12, chemokines and/or to produce iNOS, arginase or other immunomodulating enzymes;
(ii) is characterized by expression of at least one of following antigens: CD64, CD86, CD16, CD32 HLA-DR, and/or production of iNOS and/or IL-12;
(iii) is producible by in vitro incubation of a monocyte or macrophage with a factor capable of inducing the ability of the macrophage to phagocytose;
(iv) is characterized by expression of at least one of following antigens: CD204, CD206, CD200R; CCR2, transferrin receptor (TfR), CXC-motive chemokine receptor 4 (CXCR4), CD163, and/or show low expression of HLA-DR;
(v) has the ability to phagocytose; and/or
(vi) is capable of cytokine secretion, preferably of IL-12, or IL-10, or production of inducible nitric oxide synthetase (iNOS), pro-inflammatory compounds, arginase immunosuppressive compounds or anti-inflammatory compounds.

Preferably,
(i) the M1 inducer is selected from the group consisting of LPS, INF-γ, GM-CSF, and viral or bacterial proteins or products; or
(ii) the M2 inducer is selected from the group consisting of IL-4, IL-10, IL-13, immune complex of an antigen and antibody, IgG, heat activated gamma-globulin, glucocorticosteroid, TGF-β, IL-1R, CCL-2, IL-6, M-CSF, PPARγ agonist, leukocyte inhibitory factor, adenosine, helminth or fungal proteins or products.

It is preferred that the differentiated monocyte, preferably macrophage, is characterized by expression of at least one, at least two, at least three, preferably at least four, at least five, more preferably at least six, at least seven or all of TfR, CD163, CD14, CD16, CD33, 25f9, CD172a and/or CD115 or at least one, at least two, at least three, preferably at least four, at least five, more preferably at least six, at least seven or all of TfR, CD163, CD14, CD16, CXCR4, 25f9, and/or CXCR1.

With respect to the lymphocyte, it is preferred that it is selected from the group consisting of a CD3⁺ and CD4⁺ or CD8⁺T lymphocyte, or a CD19⁺, CD20⁺, CD21⁺, CD19⁺CD20⁺, CD19⁺CD21⁺, CD20⁺CD21⁺, or CD19⁺CD20⁺CD21⁺B lymphocyte, and a natural killer (NK) cell.

In a preferred embodiment of the targeted delivery system of the present invention the lymphocyte:
(i) is obtainable from blood, spleen, or bone marrow or is producible from a CD34⁺ precursor cell as known to the skilled person and also described in the, e.g. Lefort and Kim, 2010, J Vis Exp 40:2017; Tassone and Fidler, 2012, Methods in Molecular Biology 882:351-357; Kouro et al. 2005, Current Protocols in Immunology, 66: F22F.1: 22F.1.1-22F.1.9.;
(ii) is an immunologically competent lymphocyte;
(iii) expresses antigen specific T cell receptors; and/or
(iv) is characterized by expression of at least one of the following antigens: (a) CD3 and CD4 or CD8 or (b): CD19, CD20, CD21, CD19 CD20, CD19 CD21, CD20 CD21, or CD19 CD20 CD21 antigen, and is preferably capable of producing immunoglobulins

In a particularly preferred embodiment the CD45⁺ lymphocytes is a NK cell, which
(i) is obtainable from blood, spleen or bone marrow or producible from a CD34⁺ precursor cell; and/or
(ii) is characterized by the lack of CD3 expression and expression of at least one of the following CD56⁺ and/or CD94⁺, CD158a⁺CD158f⁺CD314⁺CD335⁺.

With respect to the granulocyte, it is preferred that it is selected from the group consisting of a neutrophil, preferably a CD66b⁺ neutrophil, an eosinophil and a basophil, preferably a CD193⁺ eosinophil.

In a preferred embodiment of the targeted delivery system of the present invention the granulocyte:
(i) is obtainable from blood, spleen or bone marrow or producible from a CD34⁺ precursor cell as described, e.g. in Kuhs et al. 2015, CurrProtocImmunol 111:7.23-1-7.23.16; Coquery et al. 2012, Cytometry A 81 (9): 806-814; Swemydas and Lionakis 2013, J Vis Exp 77:50586.;
(ii) is characterized by expression of at least one of the following CD66b and/or CD193;
(iii) is a polymorphonuclear leukocyte characterized by the presence of granules in its cytoplasm; and/or
(iii) is characterized by expression of at least one of the following: TfR, CD163, TIM-2, and/or CXCR4.

The targeted delivery system for use of the present invention still provides the outlined advantages, if in a population of cells not every cell has a particular property in as long as the majority of cells within that population has that property. Thus, in the following the property of one preferred cell of the targeted delivery system for use of the present invention is described.

Ways of isolating, culturing and activating the CD45+ leukocyte cells comprised in the claimed delivery system are described in WO 2016/207257 A1, WO 2016/207256 A1 and WO 2017/222398 A1.

In some embodiments, the CD45⁺ leukocyte cells, preferably macrophages, comprised in the targeted delivery system, are derived from isolated peripheral blood mononuclear cells (PBMCs). Preferably, the CD45⁺ leukocyte cells, preferably macrophages, comprised in the targeted delivery system, are primary cells, i.e. cells isolated directly from human tissues, in particular peripheral blood. It is preferred that the CD45⁺ leukocyte cells, preferably macrophages, comprised in the targeted delivery system, are not cells of an immortalized cell line. In most preferred embodiments, the CD45⁺ leukocyte cells are bone marrow derived macrophages (BMDM) or monocyte-derived macrophages (MDM).

In some embodiments, the CD45⁺ leukocyte cells comprised in the targeted delivery system, originate from the patient to be treated. In such case the cell loaded with the complex would be autologous to the patient. It is also envisioned that patients are HLA typed prior to treatment with the targeted delivery system of the present invention and that the cell type used for a given patient is HLA matched to the patient. In these two preferred embodiments, the cell is a primary cell or derived by a low number of differentiation steps from a primary cell. Alternatively, the cell may be from an immortalized but preferably non-transformed cell line.

The blood used for isolation of CD45⁺ leukocyte cells is preferably obtained from the patient to be treated or from a healthy donor. Alternatively, the blood can be obtained from the blood bank. Use of umbilical cord blood is also considered herein.

Ways of loading the CD45⁺ leukocyte cells with complexes of an iron binding protein and an active ingredient are described in WO 2016/207257 A1, WO 2016/207256 A1 and WO 2017/222398 A1.

### Mechanism

The present invention exploits CD45⁺ leukocytes, preferably activated macrophages loaded with iron-binding proteins in a complex with a drug/prodrug or a label, as a delivery system to target ovarian cancer. The inventive delivery system delivers the active ingredient directly into the ovarian cancer cells, thereby increasing efficiency and reducing side effects. When administered intravenously, the CD45+ leukocytes, preferably activated macrophages, of the inventive targeted delivery system for use in the treatment of ovarian cancer, are able to extravasate at the site of the ovarian cancer, migrate deep into the tumor tissue and deliver the active ingredient directly into the cancer cells. The inventive targeted delivery system thus ensures a better drug distribution within the tumor tissue compared to administration of an active ingredient alone or an active ingredient in a complex with an iron binding protein but without a CD45⁺ leukocyte cell. In summary, the inventive targeted delivery system constitutes an effective delivery system of active ingredients to the entire ovarian cancer mass, enabling efficient tumor cell killing with reduced systemic exposure and reduced side effects.

The inventors observed that upon intravenous administration of the isolated targeted delivery system for use according to the invention to the animal, loaded CD45⁺ leukocytes, preferably activated macrophages, migrate to the ovarian cancer site and release the complex of iron-binding protein and active ingredient(s) into the cancer cells. The inventors demonstrate that cell-cell contact is required for efficient transfer of a complex, in particular a conjugate, comprising ferritin and an active agent from macrophages to cancer cells, and that secretion of the complex, in particular conjugate, comprising ferritin and an active agent by the macrophages and subsequent uptake by the cancer cells is not sufficient to ensure efficient delivery. This indicates that the transfer of the targeted delivery system of the invention is a direct transfer between cells, requiring cell-cell contact or at least very close proximity between the CD45⁺ leukocytes (macrophages) and the cancer cells. This direct transfer has the advantage that the active agent is delivered specifically into the target cell without increasing the extracellular concentration outside the tumor cells, thereby increasing efficacy and decreasing side effects. The method of the invention thus allows an advantageously precise administration of the active ingredient(s) to the ovarian cancer site, in particular into the ovarian cancer cells.

### Combination Therapies

In some embodiments of the isolated targeted delivery system for use according to the invention, the treatment or diagnosis of ovarian cancer comprises irradiation of the ovarian cancer prior to administration of the isolated targeted delivery system. Irradiation followed by administration of the isolated targeted delivery system provides for a synergistic effect and increases treatment efficacy.

In some embodiments, administration of the isolated targeted delivery system is combined with standard therapies for ovarian cancer treatment, including chemotherapy with taxanes.

### Pharmaceutical Composition

In another aspect, the invention provides a pharmaceutical composition for the treatment or diagnosis of ovarian cancer comprising the isolated targeted delivery system described with respect to the first aspect of the invention and a pharmaceutically acceptable carrier and/or suitable excipient(s). All embodiments described with respect to the first aspect of the invention also apply to the pharmaceutical composition.

Since the isolated targeted delivery system comprises living cells, it is preferred that carriers and excipients are chosen in such to keep the cells alive.

“Pharmaceutically acceptable” means approved by a regulatory agency of the Federal or a state government or listed in the U.S. Pharmacopeia or other generally recognized pharmacopeia for use in animals, and more particularly in humans.

The term “carrier”, as used herein, refers to a pharmacologically inactive substance such as but not limited to a diluent, excipient, surfactants, stabilizers, physiological buffer solutions or vehicles with which the pharmaceutically active substance is administered. Such pharmaceutical carriers can be liquid or solid. Liquid carrier include but are not limited to sterile liquids, such as saline solutions in water and oils, including but not limited to those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like.

“Surfactants” include anionic, cationic, and non-ionic surfactants such as but not limited to sodium deoxycholate, sodium dodecylsulfate, Triton X-100, and polysorbates such as polysorbate 20, polysorbate 40, polysorbate 60, polysorbate 65 and polysorbate 80.

“Stabilizers” include but are not limited to mannitol, sucrose, trehalose, albumin, as well as protease and/or nuclease antagonists.

“Physiological buffer solution” include but are not limited to sodium chloride solution, demineralized water, as well as suitable organic or inorganic buffer solutions such as but not limited to phosphate buffer, citrate buffer, tris buffer (tris(hydroxymethyl)aminomethane), HEPES buffer ([4 (2 hydroxyethyl) piperazino]ethanesulphonic acid) or MOPS buffer (3 morpholino-1 propanesulphonic acid). The choice of the respective buffer in general depends on the desired buffer molarity. Phosphate buffer are suitable, for example, for injection and infusion solutions.

### Method of Treatment

In another aspect, the invention provides a method of treatment or diagnosis of ovarian cancer comprising administration of the isolated targeted delivery system described with respect to the first aspect of the invention in an effective amount to a patient in need thereof. All embodiments described with respect to the first aspect of the invention also apply to the method of treatment or diagnosis of ovarian cancer.

### BRIEF DESCRIPTION OF DRAWINGS

FIG. 1: A) Diagram showing the timeline of the course of treatment in an ovarian cancer SKOV-3 xenograft model. Short lines show Paclitaxel administrations. Long lines show administrations of macrophages loaded with ferritin-drug conjugate. B) Weight of both ovaries at the end of experiment. C) Bioluminescence of mice at the end of experiment. Statistical significance was calculated using ANOVA with Dunett's multiple comparisons test. D) IVIS images of tumor-related bioluminescence of all groups of mice: control, macrophages loaded with Ft-drug, Paclitaxel—on day 21 from the start of therapy. The images show a reduction in tumor size and a prevention of metastatic spread upon treatment with macrophages loaded with Ft-drug compared to paclitaxel treatment.

FIG. 2: Representative fluorescence microscopy images of different organs of the SKOV-3 xenograft mouse model. Fluorescently labeled macrophages injected intravenously extravasate and migrate to tumor-bearing organs. Blood vessels are stained with isolectin, cell nuclei are stained with DAPI. This result shows that macrophages migrate to the ovarian cancer but not to the ovary without the tumor (control ovary). Besides ovarian cancer, they migrate also to the lungs and liver. The total magnification of the microscope is 40×. Scale-100 μm.

FIG. 3: Cytotoxic activity of macrophages loaded with Ft-drug against SKOV-3 cells in in vitro culture. The results are presented as the number of viable tumor cells after treatment versus the number of viable tumor cells in the control. Left: 48 hours; right: 72 hours. Statistical significance was calculated using ANOVA with Bonferroni multiple comparisons test ns P≥0.05; *P<0.05; **P<0.01; ***P<0.001; ****P<0.0001.

FIG. 4: Cell-cell contact is required for transfer of ferritin from macrophages to cancer cells. Ferritin transfer is analysed between cells separated by Transwell insert. THP-1 macrophages loaded with HFt-AF488 (HFt (⁺)) were seeded on Transwell insert or directly with MDA-MB-231 cancer cells growing on the 24-well plate and cultured for 24 hours. Macrophages without ferritin (HFt (−)) were used as a control. Flow cytometry analysis of cancer cell AF488 fluorescence after 24-hour co-culture with macrophages. The bar plot represents average geometric mean fluorescence of AF488 positive cancer cells. AF488 positive cancer cells are detected upon direct co-culture with macrophages, but not if separated from the macrophages by a Transwell insert, indicating that cell-cell contact is required for delivery of the conjugate of ferritin and active agent (label) to the cancer cell.

FIG. 5: Cytotoxic potential of macrophages comprising a ferritin drug conjugate in co-culture with SKOV-3 cells after 72 h. Summary of three independent experiments (3 different donors). Results show viability of cancer cells calculated as number of live cells relative to number of live cells in control groups. E:T ratios: 0.125:1; 0.25:1; 0.5:1; 1:1; 2:1. Under each graph, Area Under Curve (AUC) score was presented. Bars show mean values±SD. Activity of macrophages comprising a ferritin drug conjugate with AUC<90 is considered relevant.

FIG. 6: Cytotoxic potential of macrophages comprising a ferritin drug conjugate in co-culture with A2780 cells after 24, 48, and 72 h. Summary of three independent experiments (3 different donors). Results show viability of cancer cells calculated as number of live cells relative to number of live cells in control groups. E:T ratios: 0.05:1; 0.10:1; 0.15:1; 0.20:1; 0.25:1. Under each graph, Area Under Curve (AUC) score was presented. Bars show mean values±SD. Activity of macrophages comprising a ferritin drug conjugate with AUC<90 is considered relevant.

FIG. 7: Cytotoxic potential of macrophages comprising ferritin drug conjugate in co-culture with A2780-cis cells after 24, 48, and 72 h. Summary of three independent experiments (3 different donors). Results show viability of cancer cells calculated as number of live cells relative to number of live cells in control groups. E:T ratios: 0.05:1; 0.10:1; 0.15:1; 0.20:1; 0.25:1. Under each graph, Area Under Curve (AUC) score was presented. Bars show mean values±SD. Activity of macrophages comprising ferritin drug conjugate with AUC<90 is considered relevant.

FIG. 8: Cytotoxic potential of macrophages comprising ferritin drug conjugate in co-culture with HEY-T30 cells after 72 h. Summary of three independent experiments (3 different donors). Results show viability of cancer cells calculated as number of live cells relative to number of live cells in control groups. E:T ratios: 0.125:1; 0.25:1; 0.5:1; 1:1; 2:1. Under each graph, Area Under Curve (AUC) score was presented. Bars show mean values±SD. Activity of macrophages comprising ferritin drug conjugate with AUC<90 is considered relevant.

FIG. 9: Cytotoxic potential of macrophages comprising ferritin drug conjugate in co-culture with OVCAR-4 cells after 72 h. Summary of three independent experiments (3 different donors). Results show viability of cancer cells calculated as number of live cells relative to number of live cells in control groups. E:T ratios: 0.125:1; 0.25:1; 0.5:1; 1:1; 2:1. Under each graph, Area Under Curve (AUC) score was presented. Bars show mean values±SD. Activity of macrophages comprising ferritin drug conjugate with AUC<90 is considered relevant.

FIG. 10: Mean normalized bioluminescent signal of SKOV-3_luc tumors implanted orthotopically in female nude mice (supine view). Values shown are the mean±SEM; n=8 for all groups initially. Statistical analysis (on day 42) was carried out using a one-Way ANOVA with Tukey's post-hoc test.

FIG. 11: The Kaplan-Meier survival curve of female nude mice implanted orthotopically with SKOV-3_luc tumors. N=8 for all groups initially. Statistical analysis was carried out using log-rank (Mantel-Cox) test.

FIG. 12: Mean normalized bioluminescent signal of SKOV-3_luc tumors implanted intraperitoneally in female nude mice (supine view). Values shown are the mean±SEM; n=10 for both groups initially. Statistical analysis (on day 56) was carried out using unpaired t-test.

FIG. 13: The Kaplan-Meier survival curve of female nude mice implanted intraperitoneally with SKOV-3_luc tumors. N=10 for both groups initially. Statistical analysis was carried out using log-rank (Mantel-Cox) test.

FIG. 14: The Kaplan-Meier survival curve of female nude mice implanted intraperitoneally with A2780_luc tumors. N=8 for both groups initially. Statistical analysis was carried out using log-rank (Mantel-Cox) test.

FIG. 15: The Kaplan-Meier survival curve of female nude mice implanted intraperitoneally with A2780-cis_luc tumors. N=8 for both groups initially. Statistical analysis was carried out using log-rank (Mantel-Cox) test.

### EXAMPLE SECTION

### Example 1—Production of Ferritin-Drug Conjugate (Ft-vcMMAE)

A ferritin-drug (Ft-drug) conjugate was produced by covalently linking the anti-cancer drug to ferritin using a maleimidocaproyl-valine-citrulline-para-aminobenzoyloxycarbonyl (mc-vc-PAB) linker. The anti-cancer drug comprising a mc-vc-PAB linker) was obtained from MedChem Express (Princeton, NJ). The conjugate was prepared as follows. Human ferritin solution was adjusted to a concentration of 120 μM with reaction buffer (50 mM phosphate buffer pH 6.8, containing 0.1 mM EDTA) and conjugated with 10-fold molar excess of the anti-cancer drug comprising a mc-vc-PAB linker in the presence of 20% v/v acetonitrile solution at 4° C. overnight. Maleimide groups react efficiently and specifically with free (reduced) sulfhydryls at pH 6.5-7.5 to form stable thioether bonds. The excess drug was purified and buffer-exchanged with D-PBS using PM 100 ultrafiltration concentrator. The yield of conjugation was approximately 80% of the total cysteines. Formation of the Ft-drug conjugate was confirmed by LC-MS analysis and by titration of residual free thiol group with p-chloromercuribenzoate. The concentrations of Ft-drug conjugates were determined by UV-vis spectroscopy analysis.

### Example 2—Production of Macrophages Comprising a Ferritin-Drug Conjugate

Ft-drug was loaded into macrophages by incubating bone marrow derived macrophages (BMDM) or monocyte-derived macrophages (MDM) for 1-4 hours in Ft-drug solution having a concentration from 0.5 mg/ml to 1 mg/ml in standard culture conditions. The macrophages were M0 macrophages or macrophages mildly polarized towards M2 cultured during differentiation period with M-CSF1. Macrophages comprising a ferritin-drug conjugate are also referred to as “macrophage ferritin drug conjugate” herein.

### Example 3—Treatment of SKOV-3 Mouse Model Tumors with Macrophages Loaded with a Ferritin-Drug Conjugate

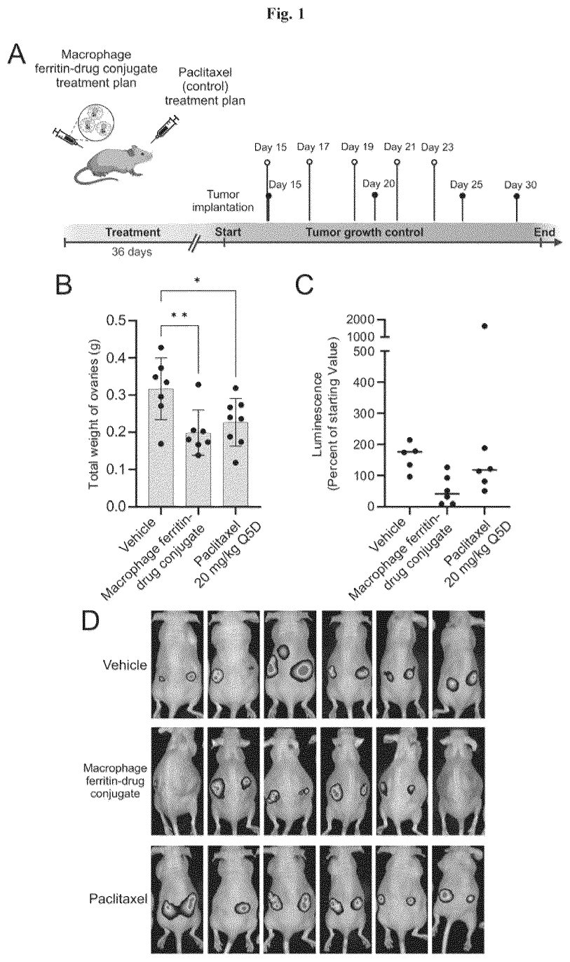

In a platinum resistant mouse ovarian cancer model (SKOV-3 xenograft mouse model), intravenous injection of monocyte-derived macrophages comprising a ferritin-drug conjugate was performed as indicated in FIG. 1A (5 million per injection). As control, injection of a vehicle was performed. As further control, injection of paclitaxel was performed as indicated in FIG. 1A (20 mg/kg per injection).

As can be seen in FIG. 1 B) to D), injection of macrophages comprising a ferritin-drug conjugate significantly reduces the ovary tumor burden and protects from metastatic spread in platinum-resistant ovarian cancer. These results demonstrate that the targeted delivery system of the invention efficiently targets and kills ovarian cancer cells. The treatment is more effective than the paclitaxel treatment, which is an established treatment in the SKOV-3 xenograft mouse model.

### Example 4—Cytotoxic Activity of Macrophages Loaded with a Ferritin-Drug Conjugate Against SKOV-3 Cells in In Vitro Culture

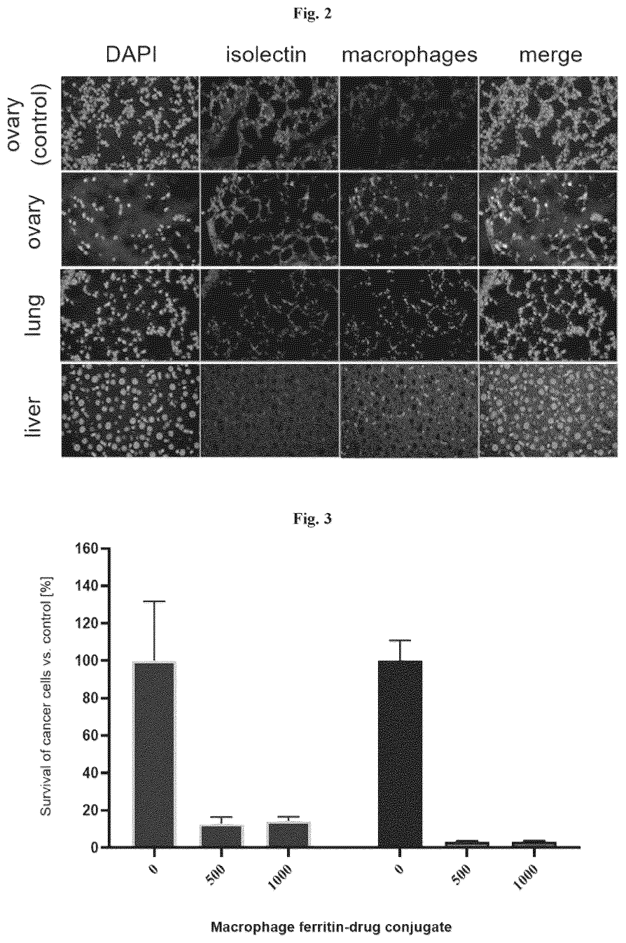

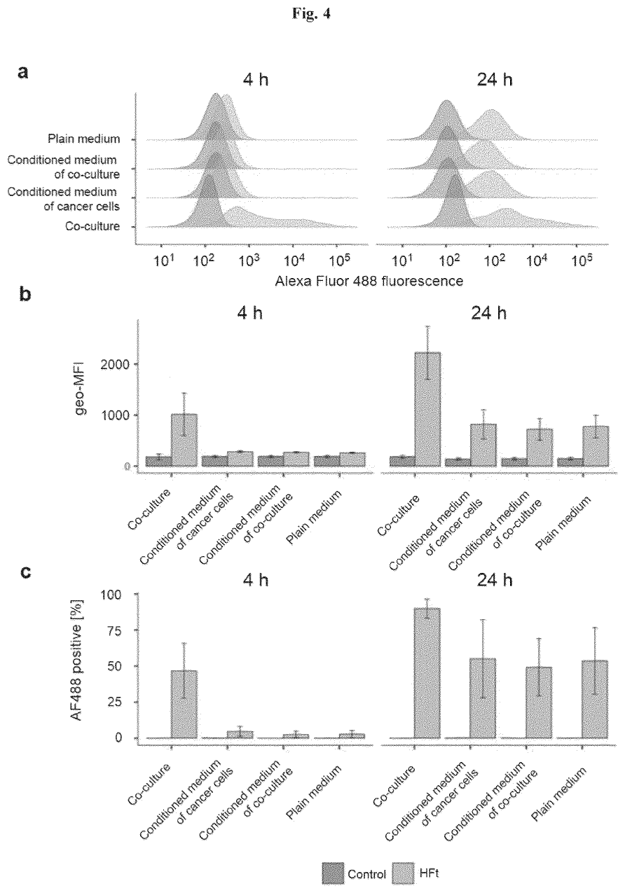

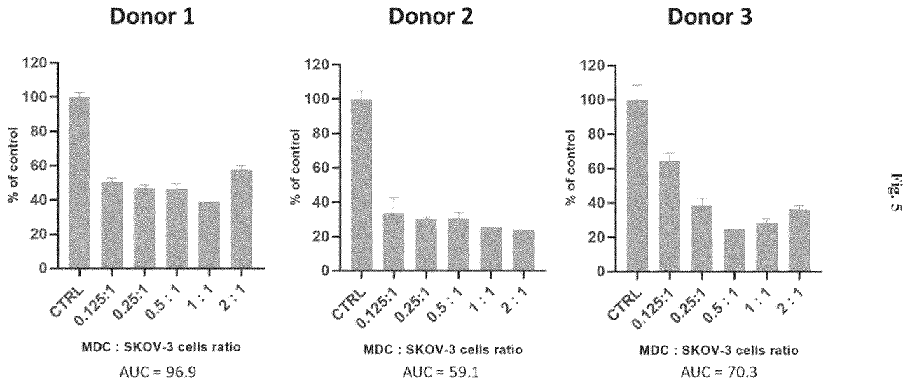

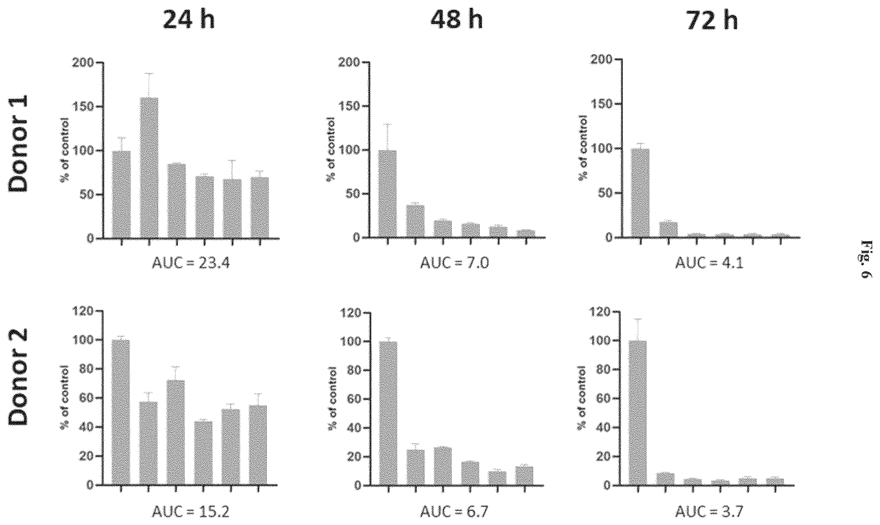

SKOV-3 cells were treated in vitro with monocyte-derived macrophages loaded with Ft-drug at the concentration of 500 or 1000 μg/ml. Viable tumor cell numbers were assessed after 48 hours and 72 hours. At all concentrations and time points, the SKOV-3 cells were efficiently killed by the treatment (FIG. 3).

### Example 5—Anti-Cancer Efficacy In Vitro

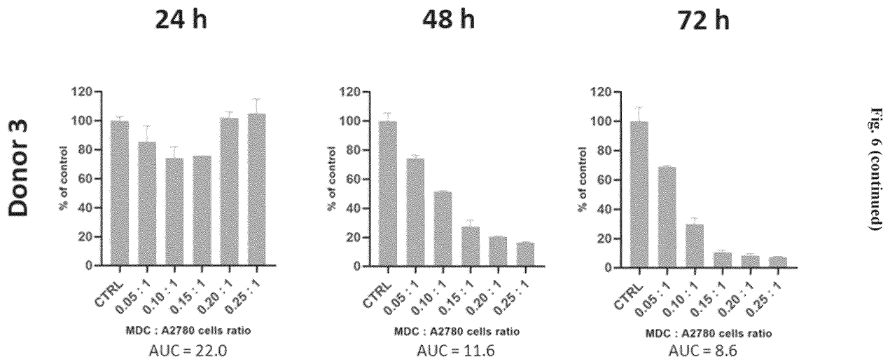

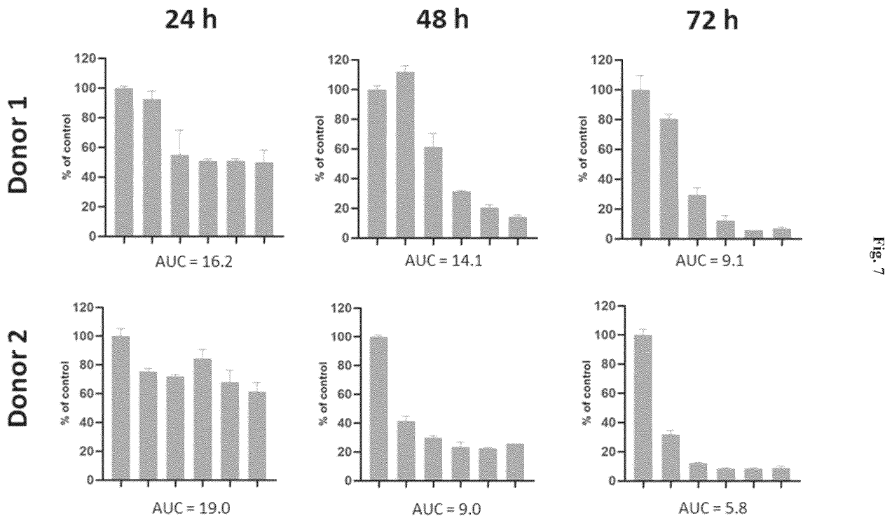

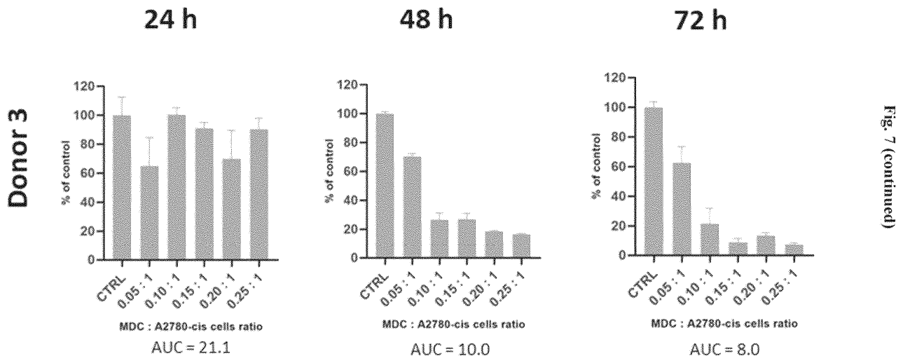

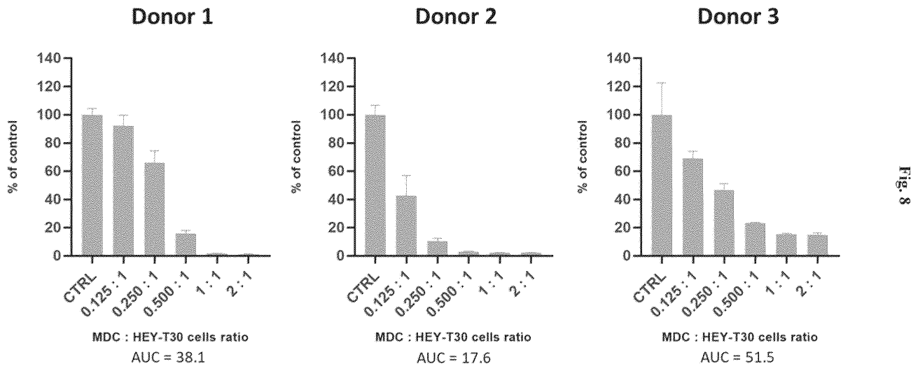

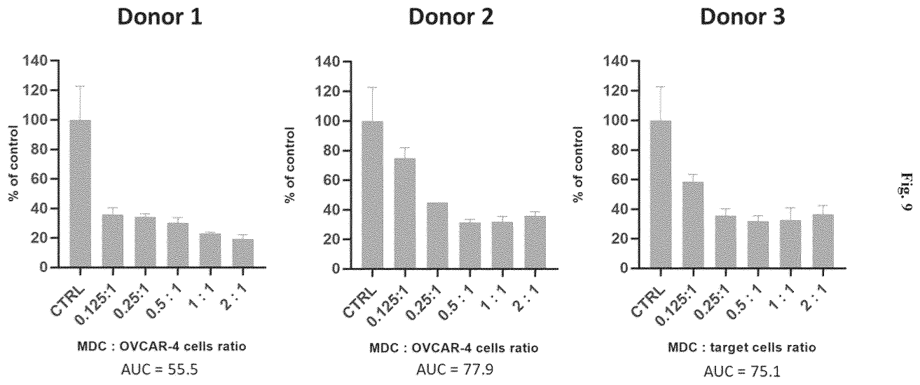

Macrophages comprising a ferritin-drug conjugate were seeded on 24-well plates with SKOV-3 or A2780 or A2780-cis (platinum-resistant) or HEY-T30 (paclitaxel-resistant) cells in different E:T ratios. Cells were incubated for 72 h, detached, and analyzed for viability using flow cytometry (FIG. 5-9).

All ovarian cancer cell lines were sensitive to the treatment: SKOV-3 and OVCAR-4 cells (AUC: 59.1-96.9 and 55.5-77.9, respectively), HEY-T30 (paclitaxel-resistant) (AUC: 17.6-51.5) and A2780 cells (A2780 AUC: 3.7-8.6; A2780-cis AUC: 5.8-9.1) (readout after 72 h). A2780 and A2780-cis (platinum-resistant) cells at lower E:T ratios responded faster (at 48 h).

### Example 6—Anti-Cancer Efficacy In Vivo

All in vivo studies were performed using female Balb/c nude mice at age 5-8 weeks. Animals were imaged using an In Vivo Imaging System (IVIS, Newton 7.0, Vilber).

### 1^{st} In Vivo Study

SKOV-3_luc cells were orthotopically implanted into both the left and right ovary of mice. Animals were imaged 14- and 21-days post-implantation to confirm the presence of tumor. Once tumor establishment on day 21 was confirmed (day 0: first treatment), mice were randomly allocated into the following treatment groups to ensure an equal spread of bioluminescence imaging (BLI) and treatment commenced:

| | Number of | |
|---|---|---|
| Treatment group | animals | Dosing Schedule |
| Vehicle (PBS) | 8 | IP; 4 times every 2-3 days |
| Macrophages comprising | 8 | IP; 4 times every 2-3 days |
| ferritin drug conjugate | | |
| (1 × 10⁷) | | |
| Trastuzumab | 8 | IV; 20 mg/kg BIW, biweekly |
| | | for 4 weeks (total 8 doses) |
| Macrophages comprising | 8 | IP; 4 times every 2-3 |
| ferritin drug conjugate | | days + IV; 20 mg/kg |
| (1 × 10⁷) + Trastuzumab | | BIW, biweekly for 4 weeks |
| | | (total 8 doses) |

Mice were imaged once weekly until day 56 (9 times on days: 0, 7, 14, 21, 28, 35, 42, 49, 56 post-treatment).

### Summary

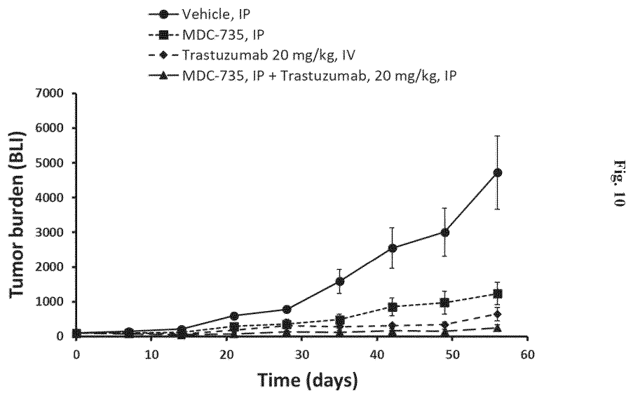

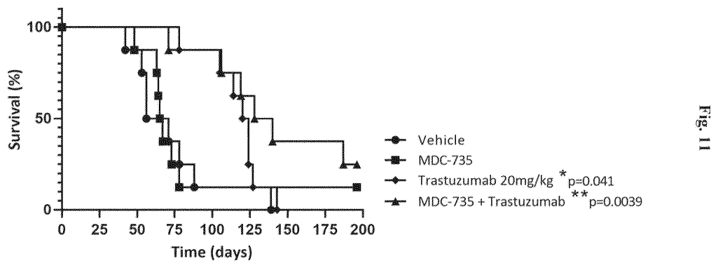

Considering supine images, on day 42 the treatments resulted in BLI values that were significantly smaller (Macrophages comprising ferritin drug conjugate: p=0.074; Trastuzumab: p=0.0003; Macrophages comprising ferritin drug conjugate +Trastuzumab: 0.0002) than those from animals treated with vehicle only (FIG. 10). Treatment with trastuzumab in combination with macrophages comprising ferritin drug conjugate resulted in a significant right-shift of the survival curve (higher median survival: 134 days, p=0.0039; FIG. 11).

### 2^{nd} In Vivo Study

SKOV-3_luc cells were intraperitoneally implanted into mice. 2-4 hours after implantation, mice were randomly allocated into the following treatment groups and dosed on a single occasion:

| | Number of | |
|---|---|---|
| Treatment group | animals | Dosing Schedule |
| Vehicle (PBS) | 10 | IP; single treatment |
| Macrophages comprising | 10 | IP; single treatment |
| ferritin drug conjugate (1 × 10⁷) | | |

Animals were imaged once weekly until day 84 (11 times on days: 0, 7, 14, 21, 28, 35, 42, 49, 56, 70, 84 post-tumor cell implantation and post-treatment).

### Summary

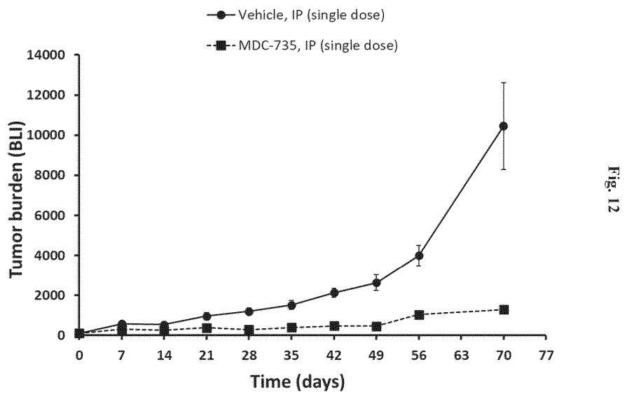

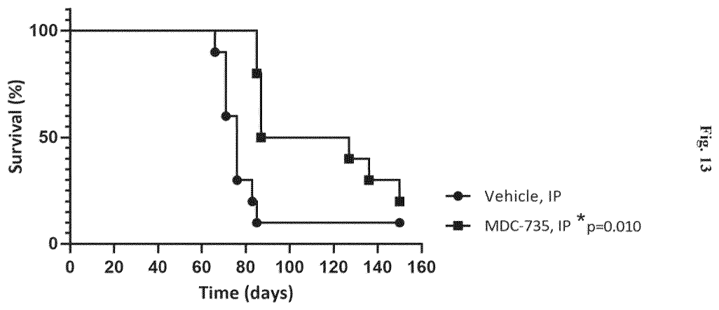

Considering supine images, on day 56 the treatment with macrophages comprising ferritin drug conjugate resulted in significantly smaller BLI values (p<0.0001) than those from control animals (FIG. 12). Treatment with macrophages comprising ferritin drug conjugate resulted in a significant right-shift of the survival curve (higher median survival: 100 days versus 76 days for vehicle, p=0.010; FIG. 13).

### 3rd In Vivo Study

A2780_luc or A2780-cis_luc cells were orthotopically implanted into both ovaries of mice. 5 days after implant mice were imaged using IVIS and those with most similar bioluminescent intensity values were assigned to the following treatment groups:

| | Number of | |
|---|---|---|
| Treatment group | animals | Dosing schedule |
| Vehicle (PBS) | 8 | IP; 4 times every 2-3 days |
| Cisplatin | 8 | IP, 3 mg/kg QW; 5, 12, 19 and 26 post-implant |
| Macrophages comprising | 8 | IP; 4 times every 2-3 days |
| ferritin drug conjugate (1 × 10⁷) | | |
| Paclitaxel | 8 | IP; 20 mg/kg QW; 5, 12, 19 and 26 post-implant |
| Macrophages comprising | 8 | IP; 4 times every 2-3 days + IP; 20 mg/kg QW; |
| ferritin drug conjugate (1 × 10⁷) + | | 5, 12, 19 and 26 post-implant |
| Paclitaxel | | |

### Summary

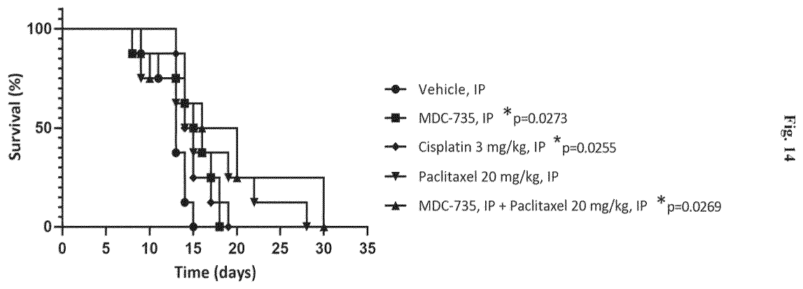

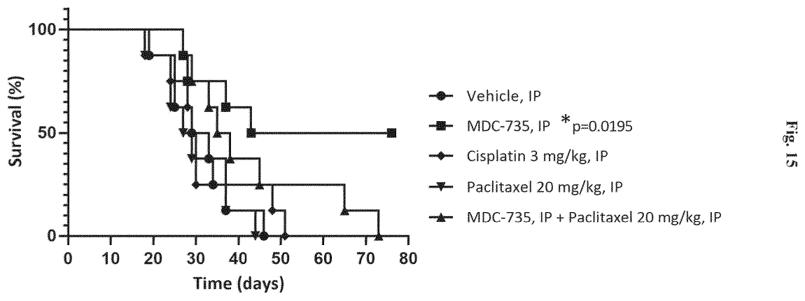

In A2780_luc orthotopic xenograft model, treatment with cisplatin, macrophages comprising ferritin drug conjugate and macrophages comprising ferritin drug conjugate combined with paclitaxel resulted in a significant right-shift of the survival curve (higher median survival: 14.5 days for cisplatin, 15.5 days for macrophages comprising ferritin drug conjugate and 18 days for macrophages comprising ferritin drug conjugate plus paclitaxel versus 13 days for vehicle, p=0.0255; p=0.0273 and p=0.0269 respectively; FIG. 14). In platinum-resistant A2780-cis_luc orthotopic xenograft model, only treatment with macrophages comprising ferritin drug conjugate resulted in a significant right-shift of the survival curve (higher median survival: 59 days for macrophages comprising ferritin drug conjugate (4 of 8 cured mice) versus 31 days for vehicle, p=0.0195; 4 out of 8 mice cured; FIG. 15).

SEQUENCES

SEQ ID NO: 1-human ferritin heavy chain
MTTASTSQVRQNYHQDSEAAINRQINLELYASYVYLSMSYYFDRDDVAL
KNFAKYFLHQSHEEREHAEKLMKLQNQRGGRIFLQDIKKPDCDDWESGL
NAMECALHLEKNVNQSLLELHKLATDKNDPHLCDFIETHYLNEQVKAIK
ELGDHVTNLRKMGAPESGLAEYLFDKHTLGDSDNES
SEQ ID NO: 2-mouse ferritin heavy chain
MTTASPSQVRQNYHQDAEAAINRQINLELYASYVYLSMSCYFDRDDVAL
KNFAKYFLHQSHEEREHAEKLMKLQNQRGGRIFLQDIKKPDRDDWESGL
NAMECALHLEKSVNQSLLELHKLATDKNDPHLCDFIETYYLSEQVKSIK
ELGDHVTNLRKMGAPEAGMAEYLFDKHTLGHGDES
SEQ ID NO. 3-mammalian consensus ferritin
MTTASXSQVRQNYXQXSEAAXXRQINLELXASYVYLSMSXYFDRDDVAL
KNFAKYFLHQSHEEREHAEKLMKLQNQRGGRIXLXDIKKPDXDDWESGL
NAMECALXLEKXVNQSLLELHKLATDKNDPHLCDFIETXYLXEQVKXIK
ELGDHVTNLRKMGAPEXGXAEYLFDKHTLGXSDXXX
SEQ ID NO. 4-FT_2
MTTASTSQVRENYHEDSEAAINRQINLELYASYVYLSMSYYFDRDDVAL
KNFAEYFLHQSHEEREHAEKLMELQNQRGGRIFLQDIQKPDCDDWESGL
NAMECALHLEKNVNQSLLELHKLATDKNDPHLCDFIETHYLNEQVEAIK
ELGDHVTNLRKMGAPESGLAEYLFDKHTLGDSDNES
SEQ ID NO. 5-alternative mammalian consensus
ferritin
MTTASXSQVRQNYXQXSEAAXXRQINLELXASYVYLSMSXYFDRDDVAL
KNFAKYFLHQSHEEREHAEKLMKLQNQRGGRIXLXDIKKPDXDDWESGL
NAMECALXLEKXVNQSLLELHKLATDKNDPHLCDFIETXYLXEQVKXIK
ELGDHVTNLRKMGAPEXGXAEYLFDKHTLGXSDXXX

1. A method of treating or diagnosing ovarian cancer, comprising administration of an isolated targeted delivery system to a patient in need thereof, wherein the isolated targeted delivery system comprises a CD45⁺ leukocyte cell comprising within said cell a complex of one or more iron binding proteins and a pharmaceutically active substance, label or pharmaceutically active substance and label.

2. The method according to claim 1, wherein
(i) the iron binding protein and the pharmaceutically active substance or label are covalently and/or non-covalently linked, and/or
(ii) the pharmaceutically active substance or label is encapsulated by the iron binding protein or multimers thereof.

3. The method according to claim 1, wherein the iron binding protein and the pharmaceutically active substance or label are covalently linked.

4. The method according to claim 3, wherein the iron binding protein and the pharmaceutically active substance or label are covalently linked via a cleavable linker.

5. The method according to claim 1, wherein the iron binding protein is conjugated to the pharmaceutically active substance, label or linker via a cysteine residue or a lysine residue.

6. The method according to claim 1, wherein the CD45⁺ leukocyte is selected from the group consisting of a monocyte, a differentiated monocyte, a lymphocyte and a granulocyte.

7. The method according to claim 6, wherein
(i) the monocyte is a CD11b⁺ monocyte;
(ii) the differentiated monocyte is selected from the group consisting of a macrophage and an activated macrophage, and a dendritic cell (DC);
(iii) the lymphocyte is selected from the group consisting of a CD3⁺ and CD4⁺ or CD8⁺ T lymphocyte, or a CD19⁺, CD20⁺, CD21⁺, CD19⁺CD20⁺, CD19⁺CD21⁺, CD20⁺CD21⁺, or CD19⁺CD20⁺CD21⁺ B lymphocyte, and a natural killer (NK) cell; or
(iv) the granulocyte is selected from the group consisting of a neutrophil, an eosinophil and a basophil.

8. The method according to claim 6, wherein the monocyte or differentiated monocyte:
(i) is producible from a CD34⁺ hematopoietic precursor cell;
(ii) is producible by in vitro incubation of monocytes with at least one inducer;
(iii) is characterized by expression of at least one of the following antigens: TfR, CD163, CD14, CD16, CD33,CXCR4, 25f9, HLA-DR and/or CD115; and/or
(iv) has the ability to phagocytose.

9. The method according to claim 7, wherein the activated macrophage:
(i) is producible by in vitro incubation of a monocyte or macrophage with a factor capable of altering expression markers on macrophages;
(ii) is characterized by expression of at least one of following antigens: CD64, CD86, CD16, CD32 HLA-DR, and/or production of iNOS and/or IL-12;
(iii) is producible by in vitro incubation of a monocyte or macrophage with a factor capable of inducing the ability of the macrophage to phagocytose;
(iv) is characterized by expression of at least one of following antigens: CD204, CD206, CD200R; CCR2, transferrin receptor (TfR), CXC-motive chemokine receptor 4 (CXCR4), CD163, and/or show low expression of HLA-DR;
(v) has the ability to phagocytose; and/or
(vi) is capable of cytokine secretion.

10. The method according to claim 1, wherein the iron binding protein is selected from the group consisting of ferritin; haemoglobin-haptoglobin complex, hemopexin, transferrin, and lactoferrin.

11. The method according to claim 1, wherein the pharmaceutically active substance is an anticancer drug selected from the group consisting of a protein, a peptide, a nucleic acid, a non-protein non-nucleic acid compound with a molecular weight of less than 1.5 kD, a photosensitizing compound, a virus, and pharmaceutically active radioactive isotope.

12. The method according to claim 11, wherein the anti-cancer drug
is selected from the group consisting of an apoptosis-inducing drug, an alkylating substance, anti-metabolites, antibiotics, an antimitotic agent, a DNA-modifying drug, a DNA minor groove interstrand crosslinking drug, an inhibitor of DNA synthesis, an inhibitor of RNA synthesis, epothilones, nuclear receptor agonists and antagonists, an anti-androgene, an anti-estrogen, a platinum compound, a hormone, a antihormone, an interferon, an inhibitor of cell cycle-dependent protein kinases (CDKs), an inhibitor of cyclooxygenases and/or lipoxygenases, a biogenic fatty acid, a biogenic fatty acid derivative, including prostanoids and leukotrienes, an inhibitor of protein kinases, an inhibitor of protein phosphatases, an inhibitor of lipid kinases, a platinum coordination complex, an ethyleneimine, a methylmelamine, a triazine, a vinca alkaloid, a pyrimidine analog, a purine analog, an alkylsulfonate, a folic acid analog, an anthracendione, a substituted urea, and a methylhydrazin derivative, an ene-diyne antibiotic, a maytansinoid, auristatin or an auristatin derivate, an immune check-point inhibitor, and an inhibitor of a tumour-specific protein or marker, a tubulin inhibitor, or a topoisomerase inhibitor;
is selected from the group consisting of acediasulfone, aclarubicine, ambazone, aminoglutethimide, L-asparaginase, auristatin, azathioprine, banoxantrone, bendamustine, bleomycin, busulfan, calcium folinate, carboplatin, carpecitabine, carmustine, celecoxib, chaliceamycin, chlorambucil, cis-platin, cladribine, cyclophosphamide, cytarabine, dacarbazine, dactinomycindapsone, daunorubicin, deruxtecan, dibrompropamidine, diethylstilbestrole, docetaxel, doxorubicin, dolastatin 10, dolastatin 15, dynemycinA, enediynes, epirubicin, epothilone B, epothilone D, estramucin phosphate, estrogen, ethinylestradiole, etoposide, exatecane derivative, flavopiridol, floxuridine, fludarabine, fluorouracil, fluoxymesterone, flutamidefosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon α, irinotecan, leuprolide, lomustine, lurtotecan, mafenide sulfate olamide, maytansine, mechlorethamine, medroxyprogesterone acetate, megastrolacetate, melphalan, mepacrine, mercaptopurine, mertansine, methotrexate, metronidazole, mitomycin C, mitopodozide, mitotane, mitoxantrone, mithramycin, nalidixic acid, neocazinostatin,nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, pyrrolobenzodiazepine,raltitrexed, rapamycin, rofecoxib, rosiglitazone, salazosulfapyridine, scriflavinium chloride, semustinestreptozocine, sn-38, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, tinidazole, topotecan, triaziquone, treosulfan, trimethoprim, trofosfamide, UCN-01, vinblastine, vincristine, vindesine, vinblastine, vinorelbine, and zorubicin;
is an immunomodulatory drug that activates or inhibits an activity of an immune cell; and/or
is a proliferation inhibiting protein or peptide.

13. The method according to claim 11, wherein the anti-cancer drug is auristatin or deruxtecan.

14. The method according to claim 1, wherein the pharmaceutically active substance is
a hypoxia-activated prodrug, or
an antigen or a nucleic acid encoding an antigen.

15. The method according to claim 1, wherein the label is selected from the group consisting of a fluorescent dye, a fluorescence emitting isotope, a radioisotope, a detectable polypeptide or nucleic acid encoding a detectable polypeptide and a contrast agent or wherein the label comprises a chelating agent which forms a complex with divalent or trivalent metal cations.

16. The method according to claim 1, wherein the ovarian cancer is resistant to other therapies.

17. The method according to claim 16, wherein the ovarian cancer is resistant to treatment with platinum and/or paclitaxel.

18. The method according to claim 1, wherein the CD45⁺ leukocyte is a macrophage.

19. The method according to claim 18, wherein the macrophage is an M0 macrophage or a macrophage that is mildly polarized towards M2.

20. The method according to claim 16, wherein the ovarian cancer is resistant to chemotherapies.


SEQUENCE LISTING (5 sequences)

SEQ ID NO: 1 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - protein, 182 aa, Mus musculus
MTTASPSQVRQNYHQDAEAAINRQINLELYASYVYLSMSCYFDRDDVALKNFAKYFLHQSHEEREHAEKLMKLQNQRGGRIFLQDIKKPDRDDWESGLNAMECALHLEKSVNQSLLELHKLATDKNDPHLCDFIETYYLSEQVKSIKELGDHVTNLRKMGAPEAGMAEYLFDKHTLGHGDES

SEQ ID NO: 3 - protein, 183 aa, unidentified
  VARIANT 180..183: note = X is any amino acid
MTTASXSQVRQNYXQXSEAAXXRQINLELXASYVYLSMSXYFDRDDVALKNFAKYFLHQSHEEREHAEKLMKLQNQRGGRIXLXDIKKPDXDDWESGLNAMECALXLEKXVNQSLLELHKLATDKNDPHLCDFIETXYLXEQVKXIKELGDHVTNLRKMGAPEXGXAEYLFDKHTLGXSDXXX

SEQ ID NO: 4 - protein, 183 aa, unidentified
MTTASTSQVRENYHEDSEAAINRQINLELYASYVYLSMSYYFDRDDVALKNFAEYFLHQSHEEREHAEKLMELQNQRGGRIFLQDIQKPDCDDWESGLNAMECALHLEKNVNQSLLELHKLATDKNDPHLCDFIETHYLNEQVEAIKELGDHVTNLRKMGAPESGLAEYLFDKHTLGDSDNES

SEQ ID NO: 5 - protein, 183 aa, unidentified; identical to SEQ ID NO: 3
  VARIANT 180..183: note = X is any amino acid